(19) United States
(12) Patent Application Publication
(10) Pub. No.: US 2006/0177379 A1
(43) Pub. Date: Aug. 10, 2006

(54) Composition comprising an agent providing a signal, an implant material and a drug

(76) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(21) Appl. No.: 11/322,694

(22) Filed: Dec. 30, 2005

Related U.S. Application Data
(60) Provisional application No. 60/640,794, filed on Dec. 30, 2004.

(51) Int. Cl. A61K 49/10; A61K 49/04; A61F 2/06

(52) U.S. Cl. 424/9.3; 424/9.4; 623/1.11

(57) ABSTRACT

The present invention relates to compositions or combinations of materials for non-degradable and degradable implantable medical devices with regard to the setup of their signal generating properties and control of their therapeutic effectiveness, as well as to a method for the control of degradation of degradable or partially degradable medical devices composed like this, based on their signal generation, and to a method for supervision of their therapeutic effectiveness and/or the release of therapeutically active ingredients from such devices.

### CROSS-REFERENCE TO RELATED APPLICATION(S)

This application claims priority from U.S. patent application Ser. No. 60/640,794, filed Dec. 30, 2004, the entire disclosure of which is incorporated herein by reference.

### BACKGROUND OF THE INVENTION

Ultra-short term implants, short term implants such as orthopedic-surgical screws, plates, nails or catheters and injection needles, as well as long term implants like joint prostheses, artificial heart valves, vascular prostheses, stents, and subcutaneous or intramuscular types of implants are manufactured from different types of materials, which are selected according to their specific biochemical and mechanical properties. These materials should be suitable for permanent use in the body, must not release toxic materials and should have specific mechanical and biochemical properties. The manufacture of such implants with new materials is increasingly allowing the functionality of the implants to be improved. In particular in this respect, systems are used which are partially degradable/dissolvable or completely (bio-)degradable.

A significant problem with such implants is that with the use of new materials limited physical properties are provided. For example, in the application of medical imaging methods for follow-up or control of the correct anatomical position or for other diagnostic or therapeutic reasons, the radiopaque or diamagnetic, paramagnetic, super paramagnetic or ferromagnetic properties may be inadequate. In particular, biodegradable materials such as polylactonic acid and its derivatives, collagens, albumin, gelatin, hyaluronic acid, starch, cellulose and the like are typically radiolucent. This also applies for example to polymers like polyurethanes, poly(ethylene vinyl acetate), silicones, acrylic polymers like polyacrylic acids, polymethylacrylic acid, polyacrylcyanoacrylate; polyethylene, polypropylene, polyamide, poly(ester urethane), poly(ether urethane), poly(ester urea), polyethers like polyethylene oxide, polypropylene oxide, pluronics, polytetramethylene glycol; vinyl polymers like polyvinylpyrrolidone, poly(vinyl alcohol), poly(vinylacetatephthalate); and parylenes, which based on material properties are excellently suited for biomedical applications. In particular, these materials are also well-suited for non-resorbable medical implants, which consist of polymers or composite materials and are primarily only weakly radiopaque or are radiolucent.

In contrast, there are other requirements of materials which are exposed to diagnostics using magnetic resonance imaging tomography methods. Unlike conventional X-ray diagnostics, which are based on the application of ionizing radiation, magnetic resonance imaging tomography (MRI) is not based on ionizing radiation but instead on the production of a magnetic field, radio-frequency energy, and magnetic field gradients. The signals produced are based predominantly on the measured relaxation times T1 (longitudinal) and T2 (transversal) of excited protons and the proton density in the tissues. So, typically, contrast materials are applied in order to, for example, influence the proton densities and/or relaxation times produced in tissues or tissue sections, such as the T1, T2, or proton densities.

Another problem is that implantable medical devices are typically modified to improve their imaging properties. For example, radiopaque fillers are often added to polymeric materials in order to improve their visibility. In connection with this, typical fillers employed are BaSO₄, bismuth sub carbonate or metals like tungsten, or other bismuth salts like bismuth sub nitrate and bismuth oxide [see, e.g., U.S. Pat. No. 3,618,614]. Other types of modifications can include the incorporation of halogenated compounds or groups into the polymer matrix. Examples of this approach are described in U.S. Pat. Nos. 4,722,344, 5,177,170, and 5,346,981.

Disadvantages of such fillers include, for example, that fundamental material properties such as the optical properties, mechanical strength, flexibility, acid and alkali resistance may be altered. Another disadvantage of the methods described above is that a minimum amount of radiopaque fillers or halogenated components must generally be added in order to produce any significant radiopaque properties, however the solubility of such filler materials in the polymer precursors is limited.

Comparable problems exist for metal-based implant materials and intravascular devices, which are in the body temporarily or permanently. Typical of such devices are stents, which often are made of metal. The use of stents is a necessarily invasive method wherein it is of significant clinical importance that the stent be positioned correctly. To achieve this, visualization by means of an image forming method, e.g. an X-ray based method, both during and after the application is customary. Based on the alloys used and the low material weights, with thin walls or low material strengths, the visibility is only weak when it exists at all. Certain conventional radiopaque components that absorb ionizing radiation, including metal alloys that are biocompatible, can be employed. However use of these typically has a negative impact on the mechanical and (bio-)chemical properties. Other conventional methods are based on the application of band markers, which are pressed on, glued on, or electrochemically deposited radiopaque materials or metallic coatings.

Among the disadvantages of such solutions are that the band markers may become dislodged or completely detached during the application, such that that they damage the tissue of the inner wall of the vessel mechanically and traumatize the surrounding tissue, especially if they are sharp-edged or are attached at the outer edges of the implant. In a possible worst case, band markers may cause complications which can render the implant useless. Moreover, such band markers can create rough surfaces which may lead to development of thromboses later on.

Other conventional methods utilize metal-based coatings that can be produced by CVD, PVD or electrochemical methods. However, in order to be able to obtain useful radiopaque coatings, the coating thicknesses necessary to produce adhesion onto the metallic substrates may not satisfy the mechanical demands put on such implants, and thus may not ensure the safety and effectiveness of such an implant.

Also, electrochemical methods used to apply metallic coatings are of only limited suitability, since the deposition of such coatings is typically associated with rough surfaces and worsening of haemo-compatibility, or, depending on the underlying substrate, the embrittlement, corrosion tendency, or other impairment of the underlying material properties of the substrate. Such limitations are well-known for titanium based alloys, whose mechanical properties—and thus functionality of the implant—deteriorates significantly as a result of embrittlement.

Ion beam assisted implantation of radiopaque materials has the disadvantage that it is extremely expensive, cost intensive, and is of only limited applicability, especially since the evaporation from the molten metal takes place in an amount that exceeds by several times the actual amount to be deposited. Also, the deposition and growth of the coating becomes irregular and difficult to control. For example, implantation of alloys from a melt is difficult to carry out in a controlled manner due to the differing evaporation rates of the individual elements.

Also known in the art are implantable medical devices that contain active ingredients in the implant body or in parts of the implant body or in coatings thereon. The active ingredients are released through complete or partial degradation of parts of the implant body or of coatings, without degradation of the implant body. Such implantable medical devices may be known to those skilled in the art as “combinatorial devices.” It is particularly desirable to control the release of the active ingredients in vivo for such devices, for both non-degradable and degradable materials which contain active ingredients.

Such conventional devices combined with active ingredients generally do not allow for an effective control of active ingredient release from outside the body, since the active ingredients used do not themselves have at their disposal any signal generating properties. In addition, if the materials in which the active ingredients are embedded are degradable or dissolvable in the presence of physiologic fluids, their degradation rate does not correlate with the release of active ingredients, even if the matrix materials are visible by signal detecting methods. An example of this is represented by drug-eluting stents, whose release of active ingredients is determined on the basis of costly in- vitro and in-vivo analysis in very expensive pre-clinical studies. However, in such clinical studies information on clinical usefulness of the devices can be gathered only by means of indirect parameters such as restenosis rates, wall thickening of the concerned vessel, ability to penetrate, etc., often measured months after implantation. Actual limitations to controlling active ingredient release are described in Schwart et al., Circulation. 2002; 106:1867.

There is therefore a need for medical implants which are detectable for diagnostic and therapeutic purposes—during or after their application—by image generating methods which are based on ionizing radiation, radio frequency radiation, fluorescence or luminescence, sound based methods, and the like.

In particular, there is a need for implants, visible when using image producing methods, which are completely or partially biodegradable or bio-erodible. There is also a need for implants for which the rate of degradation is controllable and observable by corresponding non-invasive measurement and detection methods, such as image producing methods used over the residence time, and which thus permit a correlation between implant effectiveness and therapeutic result based on data acquired for implantation/tissue limits and new tissue growth.

### SUMMARY OF EXEMPLARY EMBODIMENTS OF THE INVENTION

An exemplary embodiment of the present invention relates to compositions or combinations of materials for non-degradable and degradable implantable medical devices, including a configuration of their signal generating properties and control of their therapeutic effectiveness, as well as to methods for the control of degradation of degradable or partially degradable medical devices composed of such materials based on their signal generation, and to methods for monitoring of the therapeutic effectiveness of such devices and/or their release of therapeutically active ingredients.

Some exemplary embodiments of the present invention provide implants that may be made visible in image producing methods, and which preferably can be made visible at the same time in as many image producing methods as possible, where the methods may be based on different physical principles. Some exemplary embodiments of the present invention allow for control of the correct anatomical positioning of an implantable medical device in situ during their application by, e.g., conventional radiographic methods, and also for subsequent monitoring of their therapeutic effectiveness through the use of non-stressing or non-invasive detection methods such as MRI-based methods.

Other exemplary embodiments of the invention provide for assemblies of implantable medical devices that may contain therapeutically active ingredients and release them controllably, for example, through the use of degradable or partially degradable components. The extent of the degradation may be correlated with the extent of release of therapeutically active ingredients, or, for non-degradable implantable devices releasing an active ingredient, the active ingredient may be coupled to a signal producing agent such that the depletion of signal in the device or parts of the device indicate the extent of the release of the active ingredient.

In further exemplary embodiments of the present invention, a controllable release of active ingredients from an implant can be facilitated, in order to locally detect the enrichment of active ingredients into specific regions of the organism, organs, tissues or cells, especially in specific cell types. Additional exemplary embodiments of the present invention provide for methods and implantable medical devices whereby therapeutic effectiveness is controllable with or without active ingredient release using the enrichment of signal producing agents in specific regions of the organism, organs, tissues or cells, especially in specific cell types, wherein such agents may already have inherent signal generating properties, or where they may only be transformed in vivo into signal generating agents via biological mechanisms. Such exemplary embodiments may be advantageous if, for example, an implantable medical device is applied as a tissue substitute in malignant tissue and it changes after metastasis or tumor removal, and fulfills the purpose of releasing of signal generating agents. Recurrence in the immediate or communicable surroundings of the implant by means of selective enrichment, brought about for example through targeting groups, may render it visible in such altered cell or tissue types.

Additional exemplary embodiments of the present invention further provide methods that make it possible to avoid an impairment of the material composition of the implant that could occur through mixing in of detectable substances that can limit or even destroy the functionality. In some exemplary embodiments, the present invention makes available a composition or combination of materials for implantable medical devices or components thereof that are adjustable with respect to their signal generating properties. In yet further exemplary embodiments of the present invention, a composition or combination of materials for implantable medical devices is provided that can be adjusted with respect to the duration of identification, i.e. the temporal availability of detectable properties. In still further embodiments, the invention makes available a composition or combination of materials for implantable medical devices that is detectable by different measurement and detection methods.

Additionally, other exemplary embodiments of the invention provide a composition or combination of materials for implantable medical devices which allows detection of the range of release of therapeutically active ingredients by means of signal generating methods, especially the release of therapeutically active ingredients from implantable medical devices or from components of implantable medical devices, or the enrichment of active ingredients which are released from implantable medical devices or from components of implantable medical devices in certain regions of the organism, organs, or tissues, or in specific tissue or cell types.

Further exemplary embodiments of the present invention make available a composition or combination of materials for implantable medical devices that allows for control of the implant effectiveness, either through measurement and detection methods which make the implant-tissue boundaries visible, or by release of signal generating agents and/or their enrichment in specific regions of the organism, organs, or tissues, or in certain tissue or cell types, which may occur in the immediate vicinity of the implanted medical device.

Still further embodiments of the invention make available a composition or combination of materials for implantable medical devices which releases signal generating agents for diagnostic and/or therapeutic purposes after insertion of such devices into an animal or human body. In some exemplary embodiments, both signal-generating and therapeutic/diagnostic agents may be released at the same time, and these agents may further be coupled or bonded to each other.

Yet other exemplary embodiments of the present invention provide a composition or combination of materials for implantable medical devices or components thereof, which may permit setting up of signal generating properties, i.e. to control for which measurement and detection methods can detect the device or its components. Other exemplary embodiments of the presentinvention permit control of whether the release of signal generating agents and/or therapeutically active ingredients from the implantable medical device or components thereof are detectable directly, i.e. via a depletion of the signal generating agents in the device or the components of the device, or indirectly, i.e. via enrichment in certain regions of the organism, organs, tissues, or in specific tissue or cell types, or both.

In still further embodiments of the present invention, a method is provided for the control of degradation of degradable or partially degradable medical devices composed of certain compositions or combinations of materials based on their signal generation properties, and a method for supervision of their therapeutic effectiveness and/or the release of therapeutically active ingredients from such devices.

In other exemplary embodiments of the present invention, a method is provided that allows the determination of the extent of release of active ingredients from an implantable medical device or a component of an implantable medical device, and may further provide methods which allow determination of the extent of the local enrichment of active ingredients that are released from an implantable medical device or a component of an implantable medical device.

According to an exemplary embodiment of the present invention, a combination is provided comprising:
a. at least one signal generating agent, which may provide detectable signals directly or indirectly for use in a physical, chemical, and/or biological measurement or detection method;
b. at least one material used for the preparation of an implantable medical device and/or at least one component of an implantable medical device; and
c. at least one therapeutically active ingredient, which either directly or indirectly fulfills a therapeutic function in an animal or human organism and is directly or indirectly released in an animal or human organism from an implantable medical device or a component of the implantable medical device.

In another exemplary embodiment of the present invention, an implantable medical device or component thereof is provided, comprising at.least one signal-generating agent and at least one therapeutically active agent as defined below. The signal-generating agents and therapeutic agents may optionally be released at the same time from the device, after its insertion into the human or animal body.

In another exemplary embodiment of the present invention, an implantable medical device or component thereof is provided, comprising at least one signal-generating agent and at least one therapeutically active agent as defined below. The signal-generating agents and therapeutic agents may optionally be released at the same time from the device, after its insertion into the human or animal body.

In further embodiments of the present invention, a composition is provided for the manufacture of an implantable medical device comprising a first and a second signal-generating agent, each of which may provide detectable signals directly or indirectly for use in a physical, chemical and/or biological measurement or verification method, wherein the first agent is essentially undetectable by at least one measurement or verification method for which the second agent does provide a detectable signal.

Such exemplary arrangements according to the exemplary embodiments of the present invention can be used in the manufacture of implantable medical devices for insertion into the human or animal body, for drug-delivery implants and the like, including, for example, as a coating or a component of a coating of the device, or as a part of the construction material of the device itself.

In still further exemplary embodiments of the present invention, a method is provided for determining the extent of release of an active agent from a completely or partially degradable or dissolvable implantable medical device, or component thereof. The device may comprise at least one signal-generating agent that provides detectable signals directly or indirectly that may be detected using a physical, chemical and/or biological measurement or verification method, including an imaging method, and further comprises at least one therapeutically active agent that may be released in a human or animal organism, and wherein the device releases at least partially the therapeutically active agent(s) together with the signal generating agent(s) in the presence of physiological fluids, for example after insertion of the device into a human or animal body, and wherein the extent of active agent release can be determined by detecting the released signal-generating agent with the use of non-invasive imaging methods.

In further exemplary embodiments of the present invention, methods are provided for determining the extent of release of an active agent from a non-degradable implantable medical device or a component thereof, manufactured by use of a combination or arrangement of materials comprising a signal-generating agent, which leads directly or indirectly to detectable signals in a physical, chemical and/or biological measurement or verification method, e.g. in an imaging method, as well as a therapeutically active agent to be released in a human or animal organism, and wherein the extent of active agent release can be determined by detecting the released signal-generating agent with the use of exemplary non-invasive imaging methods.

Preferably, microspheres, optionally comprising metals and or drugs, intended for direct injection or incorporation into the human or animal body can be excluded from the embodiments of the present invention.

### BRIEF DESCRIPTION OF THE DRAWINGS

FIG. 1 shows an exemplary correlation between the release of paclitaxel from a coronary stent in the form of encapsulated nanoparticle adsorbed active substance and the in-vivo activity of the fluorescence color of the signal-generating agent Calcein-AM in accordance with an embodiment of the present invention.

### DETAILED DESCRIPTION OF EXEMPLARY EMBODIMENTS OF THE INVENTION

### Signal Generating Material

In accordance with an exemplary embodiment of the present invention, the signal generating material may be selected from inorganic, organic or inorganic-organic composites which are degradable, partially degradable, or non-degradable. Signal generating materials are understood to be those materials which lead to detectable signals when employing physical, chemical, and/or biological measurement and verification methods, e.g. image-producing methods. Carrying out the signal processing exclusively for diagnostic or therapeutic purposes each falls within the contemplated scope of the present invention.

Typical imaging methods may include, for example, radiographic methods, which are based on ionizing radiation such as conventional X-ray methods and X-ray based split image methods like computer tomography, neutron transmission tomography, radiofrequency magnetization such as magnetic resonance tomography, and radionuclide-based methods such as scintigraphy, Single Photon Emission Computed Tomography (SPECT), Positron Emission Computed Tomography (PET), ultrasound-based methods, or fluoroscopic methods or luminescence or fluorescence based methods such as Intravasal Fluorescence Spectroscopy, Raman spectroscopy, Fluorescence Emission Spectroscopy, Electrical Impedance Spectroscopy, colorimetry, optical coherence tomography, etc, as well as Electron Spin Resonance (ESR), Radio Frequency (RF) and Microwave Laser, and similar methods.

Signal generating agents may be metal-based compositions chosen from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, or other organometallic compounds. They may be in the form of powders, solutions, dispersions, suspensions, emulsions, and the like.

Metal-based agents may also include nanomorphous nanoparticles comprising 0-valent metals, metal oxides, or mixtures thereof. The metals or metal oxides used may also be magnetic; examples of these are—without excluding other metals—iron, cobalt, nickel, manganese, or mixtures thereof, for example iron-platinum mixtures, or as examples of magnetic metal oxides, iron oxides and ferrites.

Semiconducting nanoparticles may also be used in exemplary embodiments of the present invention. Examples of this include semiconductors from group II-VI, group III-V, or group IV. Group II-VI semiconductors include, e.g., MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples of group III-V semiconductors include, e.g., GaAs, GaN, GaP, GaSb, InGaAs, InP, InN, InSb, InAs, AlAs, AIP, AlSb, AIS, and mixtures thereof. Germanium, lead and silicon are exemplary of group IV semiconductors. Semiconductor materials used in practicing the present invention may also comprise mixtures of semiconductors from more than one group, and semiconductors from any of the groups mentioned above may be included in such mixtures.

Complex formed metal-based nanoparticles may also be used in exemplary embodiments of the present invention. Included in this class of materials are so-called Core-Shell configurations, as described explicitly by Peng et al., “Epitaxial Growth of Highly Luminescent CdSe/CdS Core/Shell Nanoparticles with Photo stability and Electronic Accessibility,” Journal of the American Chemical Society (1997), 119:7019-7029. Also included in such materials are semiconducting nanoparticles, which form a core with a diameter of 1-30 nm, or preferably of 1-15 nm, onto which other semiconducting nanoparticles crystallize in 1-50 monolayers, or preferably about 1-15 monolayers. For these materials, the core and shell may be present in any desired combinations as described above. In one exemplary embodiment, the core comprises CdSe and/or CdTe, and the shell comprises CdS and/or ZnS.

In other exemplary embodiments, the signal producing nanoparticles may have absorption properties for radiation in the wavelength regions of gamma rays up to microwave radiation. Alternatively, the nanoparticles may have the property of emitting radiation, especially in the wavelength range of 60 nm or less, wherein through corresponding selection of the particle size and diameter of the core and shell, the emission of light quanta may be selected to be within the range of about 20 to 1000 nm. Mixtures of such particles may be selected such that the mixtures emit quanta of different wavelengths if exposed to radiation themselves. In some embodiments the selected nanoparticles are fluorescent, and may further be fluorescent without quenching.

Signal producing metal-based agents may also include or be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) and the like. Based on especially pronounced magnetic moments, gadolinium (III), terbium (III), dysprosium (III), holmium (III) and erbium (III) may be preferred. Further, such metal-based agents may be radioisotopes. Examples of applicable radioisotopes include H 3, Be 10, O 15, Ca 49, Fe 60, In 111, Pb 210, Ra 220, Ra 224, and the like. Typically such ions are present as chelates or complexes, wherein compositions such as diethylenetriamine pentaacetic acid (“DTPA”), ethylenediamine tetra acetic acid (“EDTA”), or tetraazacyclododecane-N,N′,N″,N″′-tetra acetic acid (“DOTA”) may be used as chelating agents or ligands for lanthanides and paramagnetic ion compounds. Other exemplary organic complexing agents are described in Alexander, Chem. Rev. 95:273-342 (1995) and Jackels, Pharm. Med. Imag, Section III, Chap. 20, p. 645 (1990). Other chelating agents that may be used in exemplary embodiments of the present invention are described in U.S. Pat. Nos. 5,155,215, 5,087,440, 5,219,553, 5,188,816, 4,885,363, 5,358,704, 5,262,532, 5,188,816, 5,358,704, 4,885,363, and 5,219,553, and in Meyer et al., Invest. Radiol. 25: S53 (1990). Salts and chelates from the lanthanide group with the atomic numbers 57-83 or the transition metals with the atomic numbers 21-29, or 42 or 44 may be particulary useful in practicing the present invention.

Paramagnetic perfluoroalkyl-containing compounds may also be used as signal generating agents in exemplary embodiments of the present invention. Such compounds are described, for example, in German laid-open patent publications DE 196 03 033 and DE 197 29 013, and in PCT Publication No. WO 97/26017.

Signal generating agents may also comprise diamagnetic perfluoroalkyl-containing substances having the general formula R<PF>-L<II>-G<III>, wherein R<PF> represents a perfluoroalkyl group with 4 to 30 carbon atoms, L<II>is a linker, and G<III> represents a hydrophilic group. The linker L may be a direct bond, an —SO₂— group, or a straight or branched carbon chain with up to 20 carbon atoms which may be substituted with one or more of —OH, —COO<−>, —SO₃<−> groups, and/or one or more of —O—, —S—, —CO—, —CONH—, —NHCO—, —CONR—, —NRCO—, —SO₂—, —PO₄—, —NH—, —NR— groups, an aryl ring, or which may contain a piperazine, wherein R stands for a C₁ to C₂₀ alkyl group, which again may contain one or a plurality of O atoms and/or be substituted with —COO<−> or SO₃— groups.

The hydrophilic group G<III> may be selected from a mono- or di-saccharide, one or a plurality of —COO<−> or —SO₃<−>groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO₂—(CH₂)₂—OH group, a polyhydroxyalkyl chain with at least two hydroxyl groups, or one or a plurality of polyethylene glycol chains having at least two glycol units, wherein the polyethylene glycol chains are terminated by an —OH or —OCH₃— group or bysimilar linkages. Information relating to these examples is described, for example, in the German patent publication DE 199 48 651.

It may be preferred in some embodiments of the invention to choose paramagnetic metals in the form of metal complexes with phthalocyanines, such as those described in Phthalocyanine Properties and Applications, Vol. 14, C. C. Leznoff and A. B. P. Lever, VCH Ed. Specific examples thereof include octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, and octa(1,4,7,10-tetraoxaundecyl)Mn-phthalocyanine, as described in U.S. Patent Publication No. 2004214810.

Signal generating agents may also be selected from super-paramagnetic, ferromagnetic or ferrimagnetic compositions, including magnetic metal alloys. Signal generating agents comprising ferrites such as gamma iron oxide, magnetites or cobalt-, nickel- or manganese-ferrites, may be particles such as those described in PCT Publication Nos. WO83/03920, WO83/01738, WO85/02772, WO88/00060, WO89/03675, WO90/01295 and WO90/01899, and in U.S. Pat. Nos. 4,452,773, 4,675,173, and 4,770,183.

Further, magnetic, paramagnetic, diamagnetic or super paramagnetic metal oxide crystals having diameters of less than 4000 Angstroms may be used in embodiments of the invention as degradable non-organic agents. Suitable metal oxides may be selected from iron oxide, cobalt oxides, iridium oxides or the like. Such compositions provide suitable signal producing properties and may have favorable biocompatible properties or may be biodegradable. Crystalline agents of this group having diameters smaller than 500 Angstroms may also be used. These crystals may be associated covalently or non-covalently with macromolecular species and may be modified in a manner similar to the metal-based signal generating agents described above.

Further, zeolite-containing paramagnets and gadolinium-containing nanoparticles selected from polyoxometallates, preferably of the lanthanides (e.g., K₉GdW₁₀O₃₆), may also be used in other embodiments.

It may be preferable to limit the average particle size of the magnetic signal producing agents to less than about 5 μm in order to optimize the image producing properties, and it may be more preferable that the magnetic signal producing particle sizes are about 2 nm to 1 μm, or even more preferably about 5 nm to 200 nm. The super paramagnetic signal producing agents may be chosen, e.g., from the group of so-called SPIOs (super paramagnetic iron oxides) having an average particle size larger than 50 nm, or from the group of the USPIOs (ultra small super paramagnetic iron oxides) having an average particle size less than 50 nm.

In accordance with further embodiments of the invention, signal generating agents may be selected from the group of endohedral fullerenes, as described, for example, in U.S. Pat. No. 5,688,486 or PCT Publication No. WO 9315768. They may also be selected from fullerene derivatives and their metal complexes. It may be preferable to select fullerene species which comprise carbon clusters having 60, 70, 76, 78, 82, 84, 90, 96 or more carbon atoms. An overview of such species can be found in European patent EP 1331226A2.

Metal fullerenes or endohedral carbon-carbon nanoparticles with arbitrary metal-based components may also be used as signal generating agents in the present invention. Such endohedral fullerenes or endometallo fullerenes, which may contain rare earths such as cerium, neodymium, samanum, europium, gadolinium, terbium, dysprosium or holmium, may be preferred. Carbon coated metallic nanoparticles such as carbides may also be used. The choice of nanomorphous carbon species is not limited to fullerenes, since other nanomorphous carbon species such as nanotubes, onions, etc. may also be used. In another embodiment, fullerene species may be selected from non-endohedral or endohedral forms, which contain halogenated groups, including iodated groups, as described in U.S. Pat. No. 6,660,248.

In certain embodiments mixtures of such signal generating agents of different specifications may also be used, depending on the desired properties of the signal generating material. The signal producing agents used generally may have a size of about 0.5 nm to 1000 nm, preferably about 0.5 nm to 900 nm, or more preferably from about 0.7 to 100 nm. In this connection the metal-based nanoparticles can be provided as a powder or in polar, non-polar or amphiphilic solutions, dispersions, suspensions or emulsions. Nanoparticles are easily modifiable based on their large surface to volume ratios. The nanoparticles to be selected may for example be modified non-covalently by means of hydrophobic ligands, e.g. with trioctylphosphine, or be covalently modified. Examples of covalent ligands include thiol fatty acids, amino fatty acids, fatty acid alcohols, fatty acids, fatty acid ester groups or mixtures thereof, for example oleic cid and oleylamine.

In accordance with exemplary embodiments of the present invention, the signal producing agents may be encapsulated in micelles or liposomes with the use of amphiphilic components, or may be encapsulated in polymeric shells, wherein the micelles/liposomes may have a diameter of about 2 nm to 800 nm, preferably from about 5 nm to 200 nm, or more preferably from about 10 nm to 25 nm. The size of the micelles/liposomes used may be chosen to be dependent on the number of hydrophobic and hydrophilic groups, the molecular weight of the nanoparticles and the aggregation number. In aqueous solutions the use of branched or unbranched amphiphilic substances may be preferred in order to achieve the encapsulation of signal generating agents in liposomes/micelles. The hydrophobic nucleus of the micelles hereby contains in some embodiments a multiplicity of hydrophobic groups, preferably between 1 and about 200, more preferably between 1 and about 100 and even more preferably between 1 and about 30, according to the desired choice of the micelle size.

Hydrophobic groups may preferably be comprised of hydrocarbon groups or residues or silicon-containing residues, for example polysiloxane chains. Furthermore, they may be selected from hydrocarbon-based monomers, oligomers and polymers, or from lipids or phospholipids or comprise combinations thereof, especially glyceryl esters such as phosphatidyl ethanolamine, phosphatidyl choline, or polyglycolides, polylactides, polymethacrylate, polyvinylbutylether, polystyrene, polycyclopentadienylmethylnorbornene, polyethylenepropylene, polyethylene, polyisobutylene, polysiloxane. Further, hydrophilic polymers are also selected for encapsulation in micelles, including such compounds as polystyrenesulfonic acid, poly-N-alkylvinylpyridiniumhalides, poly(meth)acrylic acid, polyamino acids, poly-N-vinylpyrrolidone, polyhydroxyethylmethacrylate, polyvinyl ether, polyethylene glycol, polypropylene oxide, polysaccharides like agarose, dextrane, starches, cellulose, amylose, amylopectin, or polyethylene glycol or polyethylene imine of any desired molecular weight, which may be chosen based on the desired micelles property. Further, mixtures of hydrophobic or hydrophilic polymers or such lipid-polymer compositions may be used. In further embodiments, the polymers may be used as conjugated block polymers, wherein hydrophobic and also hydrophilic polymers or any desired mixtures thereof may be selected as components of 2-, 3- or multi-block copolymers.

Such signal generating agents encapsulated in micelles may also be functionalized, whereby linker (groups) may be attached at any desired position, preferably amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound using conventional methods. Here, biological molecules such as proteins, peptides, amino acids, polypeptides, lipoproteins, glycosaminoglycanes, DNA, RNA or similar bio molecules may be used.

Signal generating agents may be selected from non-metal-based signal generating agents, for example from the group of X-ray contrast agents, which may be ionic or non-ionic. Among the ionic contrast agents are salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy 1]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino)-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino) methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1(2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like, as well as other ionic X-ray contrast agents described in the literature, for example, in J. Am. Pharm. Assoc., Sci. Ed. 42:721 (1953), Swiss Patent No. 480071, JACS 78:3210 (1956), German patent No. 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. Nos. 2,705,726 and 2,895,988, Chem. Ber. 93:2347(1960), SA-A-68/01614, Acta Radiol. 12: 882 (1972), British Patent No. 870321, Rec. Trav. Chim. 87: 308 (1968), East German Patent No. 67209, German Patent Nos. 2050217 and 2405652, Farm Ed. Sci. 28: 912(1973), Farm Ed. Sci. 28: 996 (1973), J. Med. Chem. 9: 964 (1966), Arzheim.-Forsch 14: 451 (1964), SE-A-344166, British Patent No. 1346796, U.S. Pat. Nos. 2,551,696 and 1,993,039, Ann 494: 284 (1932), J. Pharm. Soc. (Japan) 50: 727 (1930), and U.S. Pat. No. 4,005,188.

Examples of non-ionic X-ray contrast agents that may be used in the exemplary embodiments of the present invention include metrizamide as described in German publication DE-A-2031724, iopamidol as described in Belgian publication BE-A-836355, iohexol as described in British publication GB-A-1548594, iotrolan as described in European publication EP-A-33426, iodecimol as described in European publication EP-A-49745, iodixanol as described in European publication EP-A-108638, ioglucol as described in U.S. Pat. No. 4,314,055, ioglucomide as described in Belgian publication BE-A-846657, ioglunioe as described in German publication DE-A-2456685, iogulamide as described in Belgian publication BE-A-882309, iomeprol as described in European publication EP-A-26281, iopentol as described in European publication EP-A-105752, iopromide as described in German publication DE-A-2909439, iosarcol as described in German publication DE-A-3407473, iosimide as described in DE-A-3001292, iotasul as described in European publication EP-A-22056, iovarsul as described in European publication EP-A-83964, or ioxilan as described in PCT publication WO87/00757, and the like.

In some exemplary embodiments of the present invention, agents may be selected that are based on nanoparticle signal generating agents, which after release into tissues and cells are incorporated or are enriched in intermediate cell compartments and/or may have an especially long residence time in the organism.

Such particles may be selected in some exemplary embodiments from water-insoluble agents. They may also contain a heavy element such as iodine or barium, or PH-50 as a monomer, oligomer or polymer (iodinated aroyloxy ester having the empirical formula C19H23I3N2O6, and the chemical names 6-ethoxy-6-oxohexy-3,5-bis (acetyl amino)-2,4,6-triiodobenzoate), or an ester of diatrizoic acid, an iodinated aroyloxy ester, or any combinations thereof. In such embodiments particle sizes which can be incorporated by macrophages may be used. A method related to this is disclosed in WO03039601 and agents preferred to be selected are disclosed in U.S. Pat. Nos. 5,322,679, 5,466,440, 5,518,187, 5,580,579, and 5,718,388. Nanoparticles may be used which are marked with signal generating agents such as PH-50, which accumulate in intercellular spaces and can thus make interstitial as well as extrastitial compartments visible.

Signal generating agents may be selected moreover from the group of the anionic or cationic lipids, such as those described in U.S. Pat. No. 6,808,720. These signal generating agents may comprise anionic lipids such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, and the like. Specially halogenated anionic lipids may be used, including fluorinated anionic lipids. The anionic lipids may contain cations from the alkaline earth metals beryllium (Be<+2>), magnesium (Mg<+2>), calcium (Ca<+2>), strontium (Sr<+2>) and barium (Ba<+2>), or amphoteric ions, such as aluminium (Al<+3>), gallium (Ga<+3>), germanium (Ge<+3>), tin (Sn+<4>) or lead (Pb<+2>and Pb<+4>), or transition metals such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In<+3>), tungsten (W<+2> and W<+4>), osmium (Os<+2> , Os<+3>and Os<+4>), iridium (Ir<+2> , Ir<+3> and Ir<+4>), mercury (Hg<+2>), or bismuth (Bi<+3>), and/or rare earths such as lanthanides, e.g. lanthanum (La<+3>) or gadolinium (Gd<+3>). Cations that may be used include calcium (Ca<+2>), magnesium (Mg<+2>) and zinc (Zn<+2>), and/or paramagnetic cations such as manganese (Mn<+2>) or gadolinium (Gd<+3>).

Cationic lipids may be chosen from phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters. Derivatives that may also be used in practicing the present invention include N-[1-(2,3-dioleoyloxy)propyl]-N,N,N-trimethylammonium chloride (“DOTMA”). Furthermore, synthetic cationic lipids may be used that are based on, for example, naturally occurring lipids such as dimethyldioctadecylammonium bromide, sphingolipids, sphingomyelin, lysolipids, glycolipids such as for example gangliosides GM1, sulfatides, glycosphingolipids, cholesterol and cholesterol esters or salts, N-succinyldioleoylphosphattidyl ethanolamine, 1,2,-dioleoyl-sn- glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein. Fluorinated, derivatized cationic lipids may also be used. Such compounds have been described in U.S. patent application Ser. No. 08/391,938.

Such lipids furthermore may be suitable as components of signal generating liposomes, which especially can have pH-sensitive properties, as described in U.S. Patent Publication No. 2004197392.

In accordance with the exemplary embodiments of the present invention, signal generating agents may also be selected from the group of so-called microbubbles or microballoons, which contain stable dispersions or suspensions in a liquid carrier substance. Such microbubbles or microballoons may comprise gases such as, e.g., air, nitrogen, carbon dioxide, or hydrogen; or noble gases such as helium, argon, xenon or krypton; or sulfur-containing fluorinated gases such as sulfurhexafluoride, disulfurdecafluoride or trifluoromethylsulfurpentafluoride; or, for example, selenium hexafluoride; or halogenated silanes such as methylsilane or dimethylsilane; or short-chain hydrocarbons such as alkanes, including methane, ethane, propane, butane or pentane; or cycloalkanes such as cyclopropane, cyclobutane or cyclopentane; also alkenes such as ethylene, propene, propadiene or butane; or alkynes such as acetylene or propyne. Ethers such as dimethylether may also be used in practicing the present invention, or ketones, or esters, or halogenated short-chain hydrocarbons, or any desired mixtures of the above. Gases that may be used further include halogenated or fluorinated hydrocarbon gases such as bromochlorodifluoromethane, chlorodifluoromethane, dichlorodifluoromethan, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane, or perfluorohydrocarbons such as, e.g., perfluoroalkanes, perfluorocycloalkanes, perfluoroalkenes or perfluorinated alkynes. Also usable in the exemplary embodiments of the present invention are emulsions of liquid dodecafluoropentane or decafluorobutane and sorbitol, or similar such as, for example, those described in PCT publication WO-A-93/05819.

Such microbubbles may be encapsulated in compounds having the structure R¹—X—Z; R²—X—Z; or R³—X—Z′, wherein R¹, R² and R³ comprise hydrophobic groups selected from straight chain alkylenes, alkyl ethers, alkyl thiolethers, alkyl disulfides, polyfluoroalkylenes and polyfluoroalkylethers, and the like; Z comprises a polar group such as, e.g., CO₂—M<+>, SO₃<−> M<+>, SO₄<−> M<+>, PO₃<−> M<+>, P0₄<−>M<+>2, N(R)₄<+> or a pyridine or substituted pyridine and a zwitterionic group; and finally X represents a linker which binds the polar group with the residues.

Gas-filled or in situ out-gassing microspheres having a size of less than about 1000 μm may be further selected from biocompatible synthetic polymers or copolymers which may comprise monomers, dimers or oligomers, or other pre-polymer to pre-stages of the following polymerizable substances: acrylic acid, methacrylic acid, ethyleneimine, crotonic acid, acryl amide, ethyl acrylate, methylmethacrylate, 2-hydroxyethylmethacrylate (HEMA), lactonic acid, glycolic acid, [epsilon]caprolactone, acrolein, cyanoacrylate, bisphenol A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamides, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium-2-sulfoxyethylmethacrylate, vinyl pyridine, amimoethylmethacrylate, 2-methacryloyloxy-trimethylammonium chloride, or polyvinylidenes that may also be polyfunctional cross-linkable monomers such as, for example, N,N′-methylene-bis-acrylamide, ethylene glycol dimethacrylate, 2,2′-(p-phenylenedioxy)-diethyldimethacrylate, divinylbenzene, triallylamine or methylene-bis-(4-phenyl-isocyanate), and further including any desired combinations thereof. Polymers that may be used in conjunction with the present invention may comprise polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamides (e.g. nylon) and the like, or any arbitrary mixtures thereof. Copolymers that may be chosen include polyvinylidene-polyacrylonitrile, polyvinylidene-polyacrylonitrile-polymethylmethacrylate, and polystyrene-polyacrylonitrile and the like, or any desired mixtures thereof. Methods for manufacture of such microspheres are described, for example, in U.S. Pat. Nos. 4,179,546, 3,945,956, 3,293,114, 3,401,475, 3,479,811, 4,108,806, 3,488,714, 3,615,972, 4,549,892, 4,540,629, 4,421,562, 4,420,442, 4,898,734, 4,822,534, 3,732,172, 3,015,128, and 3,594,326, Japan Kokai Tokkyo Koho 62 286534, British Patent No. 1,044,680, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964).

Other signal generating agents that can be used in accordance with exemplary embodiments of the present invention may be selected from the group of agents which are transformed into signal generating agents in organisms by means of in-vitro or in-vivo cells, cells as a component of cell cultures, of in-vitro tissues, or cells as a component of multicellular organisms, such as for example fungi, plants, or animals, including in some embodiments from mammals such as mice or humans. Such agents can be made available in the form of vectors for the transfection of multicellular organisms, wherein the vectors may contain recombinant nucleic acids for the coding of signal generating agents. In other exemplary embodiments, such signal generating agents may comprise metal binding proteins. Vectors may also be viruses such as, for example, adeno viruses, adeno virus associated viruses, herpes simplex viruses, retroviruses, alpha viruses, pox viruses, arena-viruses, vaccinia viruses, influenza viruses, polio viruses, or hybrids of any of the above.

According to another exemplary embodiment of the present invention, signal generating agents may also be chosen in combination with delivery systems, in order to incorporate nucleic acids, which may be suitable for coding for signal generating agents, into the target structure. Virus particles may also be used in some embodiments for the transfection of mammalian cells, wherein the virus particle contains one or a plurality of coding sequence/s for one or a plurality of signal generating agents as described above. In these embodiments the particles may generated from one or a plurality of viruses such as, e.g., adeno viruses, adeno virus associated viruses, herpes simplex viruses, retroviruses, alpha viruses, pox viruses, arena-viruses, vaccinia viruses, influenza viruses and polio viruses.

In further exemplary embodiments, these signal generating agents can be provided from colloidal suspensions or emulsions, which are suitable to transfect cells, including mammalian cells, wherein these colloidal suspensions and emulsions contain those nucleic acids which possess one or a plurality of the coding sequence(s) for signal generating agents. Such colloidal suspensions or emulsions may contain macromolecular complexes, nano capsules, microspheres, beads, micelles, oil-in-water- or water-in-oil emulsions, mixed micelles and liposomes, or any desired mixture of the above.

In further exemplary embodiments, cells, cell cultures, organized cell cultures, tissues, organs of desired species, and non-human organisms may be chosen which contain recombinant nucleic acids having coding sequences for signal generating agents. In certain exemplary embodiments organisms may be selected from the group that includes, but is not limited to: mouse, rat, dog, monkey, pig, fruit fly, nematode worm, fish, or plants or fungi. Further, cells, cell cultures, organized cell cultures, tissues, organs of desired species and non-human organisms, may also contain one or a plurality of vectors as described above.

Signal generating agents may be produced in vivo from the group of proteins and made available as described above. Such agents may be directly or indirectly signal producing, whereby the cells produce (direct) a signal producing protein through transfection, or alternatively produce a protein which induces (indirect) the production of a signal producing protein. These signal generating agents may be detectable in methods such as MRI, whereas the relaxation time T1, T2, or both may be altered and lead to signal producing effects which can be processed sufficiently for imaging. Such proteins may comprise protein complexes, especially metalloprotein complexes. Direct signal producing proteins that may be used in some embodiments include metalloprotein complexes which are formed in the cells. Indirect signal producing agents include proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), Transferrin receptor (for the take-up of Fe), or erythroid-5-aminobevulinate synthase (for the utilization of Fe, H-Ferritin and L-Ferritin for the purpose of Fe storage). In some exemplary embodiments, both types of signal generating agents—direct and indirect—may be combined with each other, such as e.g. an indirect signal generating agent that regulates the iron-homeostasis and a direct agent that represents a metal binding protein.

In such exemplary embodiments, where metal-binding polypeptides are selected as indirect agents, it may be advantageous if the polypeptide binds to one or a plurality of metals which possess signal generating properties. Such metals include those with unpaired electrons in the Dorf orbitals, such as Fe, Co, Mn, Ni, Gd etc., wherein it is noted that Fe may be available in high physiological concentrations in organisms. Such agents may also form metal-rich aggregates, for example crystalline aggregates, whose diameters are larger than about 10 picometers, preferably larger than about 100 picometers, more preferably larger than about 1 nm, yet more preferably larger than about 10 nm, or even more preferably larger than about 100 nm.

Metal-binding compounds may also be used which have sub-nanomolar affinities with dissociation constants of less than about 10⁻¹⁵ M, or 10⁻² M or smaller. Typical polypeptides or metal-binding proteins include lactoferrin, ferritin, or other dimetallocarboxylate proteins or the like, or so-called metal catcher with siderophoric groups, such as haemoglobin. One method that may be used for selection and preparation of such signal generating agents, including possible direct or indirect agents which are producible in vivo and are suitable as signal generating agents, is disclosed in PCT publication WO 03/075747.

Another group of signal generating agents that may be used includes photophysically signal producing agents which consist of dyestuff-peptide-conjugates. Such dyestuff-peptide-conjugates may provide a wide spectrum of absorption maxima, for example polymethin dyestuffs, in particular cyanine-, merocyanine-, oxonol- and squarilium dyestuffs. The class of polymethin dyestuffs that may be used includes the cyanine dyestuffs, e.g. the indole structure based indocarbo-, indodicarbo- and indotricarbocyanines. Such dyestuffs may be substituted with suitable linking agents and may be functionalized with other groups as desired. Information relating to this is described, e.g., in German publication DE 19917713.

In accordance with another exemplary embodiment of the present invention, signal generating agents may also be functionalized as desired. The fimctionalization by means of so-called “Targeting” groups is to be understood as finctional chemical compounds which link the signal generating agent or its specifically available form (e.g. encapsulation, micelles, microspheres, vectors, etc.) to a specific functional location, or to a determined cell type, tissue type, or other desired target structures. Targeting groups may permit the accumulation of signal-producing agents in or at specific target structures. Therefore the targeting groups may be selected from the class of substances that are suitable for providing a purposeful enrichment of the signal generating agents in their specifically available form by physical, chemical or biological routes, or by combinations thereof. Useful targeting groups may include, e.g., antibodies, cell receptor ligands, hormones, lipids, sugars, dextrane, alcohols, bile acids, fatty acids, amino acids, peptides or nucleic acids, which can be chemically or physically attached to signal-generating agents in order to link the signal-generating agents into/onto a specifically desired structure. In one exemplary embodiment, targeting groups are selected that are capable of enriching signal-generating agents in or on a tissue type, or on surfaces of cells. It is not necessary for the signal generating agent to be taken up into the cytoplasm of the cells, but this may be the case. Peptides may be used as targeting groups. For example, chemotactic peptides may be used to make inflammation reactions in tissues visible by means of signal generating agents. More information related to this is described in, e.g., PCT publication WO 97/14443.

Antibodies may also be used in accordance with the present invention, including antibody fragments, Fab, Fab2, Single Chain Antibodies (for example Fv), chimerical antibodies, and the like. Other exemplary embodiments may use antibody-like substances, for example so-called anticalines. The antibodies used in some embodiments of the present invention may be modified after preparation, recombinants may be produced, or they may be human or non-human antibodies. Humanized or human antibodies may also be used in other embodiments. Examples of humanized forms of non-human antibodies include chimerical immunoglobulines, immunoglobulin chains or fragments (such as Fv, Fab, Fab′, F(ab″)2 or other antigen-binding subsequences of antibodies, which may partly contain sequences of non-human antibodies; humanized antibodies containing, e.g., human immunoglobulines (receptor or recipient antibody), in which groups of a CDR (Complementary Determining Region) of the receptor are replaced through groups of a CDR of a non-human (spender or donor antibody), wherein the spender species (e.g., mouse, rabbit or the like) has appropriate specificity, affinity, and capacity for the binding of target antigens. In a few forms the Fv framework groups of the human immunglobulines may be replaced by means of corresponding non-human groups. Humanized antibodies can moreover contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. In accordance with the present invention, targeting groups may also be hetero-conjugated antibodies. The selected antibodies or peptides may function as, e.g., cell surface markers or molecules, particularly of cancer cells, wherein a large number of known surface structures are known such as, e.g., HER2, VEGF, CA15-3, CA 549, CA 27.29, CA 19, CA 50, CA242, MCA, CA125, DE-PAN-2, and the like.

Targeting groups may also be selected which contain the functional binding sites of ligands. Such groups may be chosen from all types that are suitable for binding to any desired cell receptors. Examples of target receptors include, but are not limited to, insulin receptors, insulin-like growth factor receptors (e IGF-1 and IGF-2), growth hormone receptors, glucose transporters (particularly GLUT 4 receptors), transferrin receptors (transferrin), Epidermal Growth Factor receptors (EGF), low density lipoprotein receptors, high density lipoprotein receptors, leptin receptors, oestrogen receptors; interleukin receptors including IL-1, IL-2, IL-3, IL-4, IL-5, IL-6, IL-7, IL-8, IL-9, IL-11, IL-12, IL-13, IL-15, and IL-17 receptors, VEGF receptors (VEGF), PDGF receptors (PDGF), Transforming Growth Factor receptors (including TGF-[alpha] and TGF-[beta]), EPO receptors (EPO), TPO receptors (TPO), ciliary neurotrophic factor receptors, prolactin receptors, and T-cell receptors.

Receptors that may be used in accordance with certain embodiments of the present invention include hormone receptors, especially those for hormones such as steroidal hormones or protein- or peptide-based hormones, including but not limited to epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands may include those which are on the cell surface receptors of hormones, lipids, proteins, glycol proteins, signal transducers, growth factors, cytokine, and other bio molecules. Moreover, targeting groups may be selected from carbohydrates with the general formula: C(H₂O)_{y}, also including monosaccharides, disaccharides and oligo- as well as polysaccharides, as well as other polymers which consist of sugar molecules that contain glycosidic bonds. Carbohydrates that may be used in accordance with this invention include those that contain glycosylated proteins, including the monomers and oligomers of galactose, mannose, fructose, galactosamine, glucosamine, glucose, sialic acid, and the glycosylated components, which make possible the binding to specific receptors, especially cell surface receptors. Other useful carbohydrates that may be selected are those that contain monomers and polymers of glucose, ribose, lactose, raffinose, fructose and other biologically occurring carbohydrates such as polysaccharides, as well as e.g. arabinogalactan, gum Arabica, mannan and the like, which are usable in order to introduce signal generating agents into cells. Information relationg to such compositions is disclosed in, e.g., U.S. Pat. No. 5,554,386. Furthermore, targeting groups may be selected from the lipid group, which includes fats, fatty oils, waxes, phospholipids, glycolipids, terpenes, fatty acids and glycerides, and triglycerides. Also included are eicosanoides, steroids, sterols, suitable compounds of which can also be hormones like prostaglandins, opiates and cholesterol, and the like.

In accordance with the invention functional groups that possess inhibiting properties may also be selected as targeting groups, such as e.g. enzyme inhibitors, preferably those which link signal generating agents into/onto enzymes.

In other exemplary embodiments, targeting groups may be selected from a group of functional compounds which make possible internalization or incorporation of signal generating agents in the cells, especially in the cytoplasm or in specific cell compartments or organelles, such as for example the cell nucleus. A targeting group may contain all or parts of HIV-1 tat-proteins, their analogs and derivatized or functionally similar proteins, which in this way allows an especially rapid uptake of substances into the cells. Example of this are described in, e.g., Fawell et al., PNAS USA 91:664 (1994); Frankel et al., Cell 55:1189, (1988); Savion et al., J. Biol. Chem. 256:1149 (1981); Derossi et al., J. Biol. Chem. 269:10444 (1994); and Baldin et al., EMBO J. 9:1511 (1990).

Targeting groups may also be selected from the so-called Nuclear Localisation Signal (NLS), wherein short positively charged (basic) domains are understood to bind to specifically targeted structures of cell nuclei. Numerous NLS and their amino acid sequences are known, including single basic NLS like that of the SV40 (monkey virus), large T Antigen (pro Lys Lys Lys Arg Lys Val) (see, e.g., Kalderon, et al., Cell, 39:499-509 (1984)), the teinoic acid receptor-[beta] nuclear localization signal (ARRRRP); NFKB p50 (EEVQRKRQKL) (see, e.g., Ghosh et al., Cell 62:1019 (1990)); NFKB p65 (EEKRKRTYE) (see, e.g., Nolan et al., Cell 64:961 (1991)), as well as others (see for example Boulikas, J. Cell. Biochem. 55(1):32-58 (1994), and double basic NLS's such as xenopus (African clawed toad) proteins, nucleoplasmin (Ala Val Lys Arg Pro Ala Ala Thr Lys Lys Ala Gly Gln Ala Lys Lys Lys Lys Leu Asp), (see, e.g., Dingwall, et al., Cell, 30:449-458 (1982) and Dingwall, et al., J. Cell Biol., 107:641-849 (1988)). Numerous localization studies have shown that NLSs, which are built into synthetic peptides which normally do not address the cell nucleus or were coupled to reporter proteins, lead to an enrichment of such proteins and peptides in cell nuclei. In this connection, exemplary references are made to descriptions of Dingwall and Laskey, Ann. Rev. Cell Biol., 2:367-390 (1986); Bonnerot, et al., Proc. Natl. Acad. Sci. USA, 84:6795-6799 (1987); Galileo, et al., Proc. Natl. Acad. Sci. USA, 87:458-462 (1990). Targeting groups may also be selected for the hepatobiliary system. Similar groups are described in, e.g., U.S. Pat. Nos. 5,573,752 and 5,582,814.

### Therapeutically Active Agents

In accordance with further exemplary embodiments of the present invention, at least one therapeutic agent may also be chosen in addition to a signal generating agent. Therapeutic agents include all substances, which develop local and/or systemic physiological and/or pharmacological effects in animals, especially in mammals, for example but not limited to domestic animals such as dogs and cats; agricultural animals like pigs, cattle, sheep, or goats; laboratory animals such as mice or rats; primates such as apes, chimpanzees, etc., and humans. Therapeutic agents used in accordance with the exemplary embodiments may be present in the composition or combination in crystalline, polymorphous or amorphous forms, or any mixtures thereof. Useful therapeutically active ingredients can be chosen from a large number of therapeutically effective substances, including but not limited to enzyme inhibitors, hormones, cytokines, growth factors, receptor ligands, antibodies, antigens, ion-binding materials, among which are also included crown ethers and other chelating agents, substantially complementary nucleic acids, nucleic acid binding proteins including transcription factors, toxins, and the like. Further useful materials include cytokines such as erythropoietin (EPO), thrombopoietin (TPO), interleukin (including IL-1 through IL-17), insulin, insulin-like growth factors (including IGF-1 and IGF-2), epidermal growth factor (EGF), transforming growth factors (including TGF-[alpha] and TGF-[beta]), human growth hormone, transferrin, epidermal growth factor (EGF), Low density lipoprotein, high density lipoprotein, leptin, VEGF, PDGF, ciliary neurotrophic factor, prolactin, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxin including ricin, and all other materials which are listed in the Physician's Desk Reference, 58^{th} Edition, Medical Economics Data Production Company, Montvale, N.J., 2004 and in the Merck Index, 13^{th} Edition (especially pages Ther-1 through Ther-29).

In certain exemplary embodiments, the therapeutically active substance may be chosen from the group of active substances used for the therapy of oncological diseases and cell or tissue changes. Useful therapeutic agents include but are not limited to anti-neoplastically active substances, including alkylating agents such as alkyl sulfonates (e.g. busulfane, improsulfane, piposulfane), aziridines (e.g. benzodepa, carboquone, meturedepa, uredepa); ethylene imines and methylmelamine (e.g. altretamine, triethylenemelamine, triethylenephosphoramide, triethylenethiophosphoramide, trimethylolmelamine); so-called nitrogen mustards (e.g. chlorambucil, chlomaphazine, cyclophosphamide, estramustine, ifosfamide, mechlorethamine, mechlorethaminoxide hydrochloride, melphalan, novembichine, phenesterine, prednimustine, trofosfamide, uracil mustard); nitrosourea compounds (carmustine, chlorozotocin, fotenmustine, lomustine, nimustine, ranimustine); dacarbazine, mannomustine, mitobranitol, mitolactol; pipobromane; doxorubicine, and cis-platin (including derivatives), or the like and their derivatives. In other embodiments the therapeutically active substance may be chosen from the group of antiviral and antibacterial active substances that includes aclacinomycine, actinomycine, anthramycine, azasenrne, bleomycin, cuctinomycine, carubicine, carzinophiline, chromomycine, ductinomycine, daunorubicine, 6-diazo-5-oxn-1-norieucine, duxorubicine, epirubicine, mitomycine, mycophenolic acid, nogalumycine, olivomycine, peplomycine, plicamycine, porfiromycine, puromycine, streptonigrine, streptozocine, tubercidine, ubenimex, zinostatine, zorubicine, the aminoglycosides or polyenes or macrolide antibiotics, and the like, or their derivates.

In one embodiment the therapeutically active substance is selected from the group of radio-sensitizer drugs.

In a further exemplary embodiment the therapeutically active substance is chosen from the group that includes both steroidal active substances as well also as non-steroidal anti-inflammatory active substances.

In yet a further exemplary embodiment, the therapeutically active substance is chosen from active substances which relate to the angiogenesis, including but not limited to endostatin, angiostatin, interferones, platelet factor 4 (PF4), thrombospondine, transforming growth factor beta, the tissue inhibitors of metalloproteinase -1, -2 and -3 (TIMP-1, -2 and -3), TNP-470, marimastate, neovastate, BMS-275291, COL-3, AG3340, thalidomide, squalamine, combrestastatin, SU5416, SU6668, IFN-[alpha], EMD121974, CAI, IL-12 and IM862, and the like, or their derivatives.

In further exemplary embodiments the therapeutically active substance is chosen from the group of nucleic acids, which also includes oligonucleotides in addition to nucleic acids, and wherein at least two nucleotides are covalently linked with each other and which may optionally produce gene therapeutic or anti sense effects. Nucleic acids used in these embodiments may contain phosphodiester linkages, including those which are present as analogs with various backbones. Analogs may also contain as backbones, for example, phosphoramides (see, e.g., Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)); phosphorothioates (see e.g. Mag et al., Nucleic Acids Res. 19:1437 (1991) and U.S. Pat. No. 5,644,048); phosphorodithioates (see, e.g., Briu et al., J. Am. Chem. Soc. 111:2321 (1989)); O-methylphosphoroamidite compounds (see, e.g., Eckstein, Oligonucleotides and Analogues: A Practical Approach, Oxford University Press); and Peptide-Nucleic Acid Backbones and their Compounds (see, e.g., Egholm, J. Am. Chem. Soc. 114:1895 (1992), Meier et al., Chem. Int. Ed. Engl. 31:1008 (1992), Nielsen, Nature, 365:566 (1993), and Carlsson et al., Nature 380:207 (1996)). Other analogs that may be used include those with ionic backbones (see, e.g., Denpcy et al., Proc. Natl. Acad. Sci. USA 92:6097 (1995)), or non-ionic backbones (see, e.g., U.S. Pat. Nos. 5,386,023, 5,637,684, 5,602,240, 5,216,141 and 4,469,863; Kiedrowshi et al., Angew. Chem. Intl. Ed. English 30:423 (1991), Letsinger et al., J. Am. Chem. Soc. 110:4470 (1988), Letsinger et al., Nucleosides & Nucleotides 13:1597 (1994), Chapter 2 and 3, ASC Symposium Series 580, “Carbohydrate Modifications in Antisense Research”, Ed. Y. S. Sanghui and P. Dan Cook, Mesmaeker et al., Bioorganic & Medicinal Chem. Lett. 4:395 (1994), Jeffs et al., J. Biomolecular NMR 34:17 (1994), and Tetrahedron Lett. 37:743 (1996)), and Non-Ribose Backbones, including ones such as those which are described in U.S. Pat. Nos. 5,235,033 and 5,034,506, and in Chapters 6 and 7, ASC Symposium Series 580, “Carbohydrate Modifications in Antisense Research”, Ed. Y. S. Sanghui and P. Dan Cook. Nucleic acids having one or a plurality of carbocyclic sugars may also be used as nucleic acids in accordance with exemplary embodiments of the present invention (see e.g. Jenkins et al., Chem. Soc. Rev. (1995) pp 169-176), as well as others such as those described in Rawls, C & E News, Jun. 2, 1997, page 35. In addition to the selection of conventional useable nucleic acids and nucleic acid analogs, any desired mixtures of naturally occurring nucleic acids and nucleic acid analogs or mixtures of nucleic acid analogs may also be used.

In one exemplary embodiment, the therapeutically active substance may be chosen from the group of metal ion complexes, such as those generally described in PCT publication US95/16377, PCT publication US95/16377, PCT publication US96/19900, or PCT publication US96/15527, wherein such agents reduce or inactivate the bioactivity of their target molecules, which may be proteins, including but not limited to enzymes.

Therapeutically active substances may also be antimigratory, antiproliferative or immuno-supressive, antunflammatory or re-endothelialising active substances, including e.g. everolimus, tacrolimus, sirolimus, mycofenolate mofetil, rapamycine, paclitaxel, actinomycine D, angiopeptine, batimastate, oestradiol, VEGF, statins, and their derivates and analogs.

Therapeutically active substances or active substance combinations may also be selected from heparin, synthetic heparin-analogs (e.g. fondaparinux), hirudin, antithrombin III, drotrecogin alpha; fibrinolytics such as alteplase, plasmine, lysokinases, factor XIIa, prourokinase, urokinase, anistreplase, streptokinase; thrombozytene aggregations inhibitors such as acetylsalicylic acid, ticlopidine, clopidogrel, abciximab, dextrane; cortico-steroids like alclometasone, amcinonide, augmented betamethasone, beclomethasone, betamethasone, budesonide, cortisone, clobetasol, clocortolone, desonide, desoximetasone, dexamethasone, flucinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; zyto-statiks such as alkaloids and podophyllum toxins such as vinblastine or vincristine; alkylating agents such as nitroso urea, nitrogen lacking analogs; zytotoxic antibiotics such as daunorubicin, doxorubicin and other anthracyclines and related substances, bleomycin, mitomycin, anti-metabolites such as folic acid, purine or pyrimidine analogs; paclitaxel, docetaxel, sirolimus; platinum compounds such as carboplatinum, cisplatinum or oxali-platinum; amsacrine, irinotecane, imatinib, topotecan, interferon alpha 2a, interferon alpha 2b, hydroxycarbamide, miltefosine, pentostatin, porfimer, aldesleucine, bexarotene, tretinoin; antiandrogenes, and antioestrogenes; antiarrythmics, including antiarrythmics of Class I-like antiarrythmics of the chinidine type, e.g., chinidine, dysopyramide, ajmaline, prajmaliumbitartrate, detajmiumbitartrate; antiarrhythmics of the lidocaine type, e.g., lidocaine, mexiletine, phenytoine, tocainide; antiarrhythmics of Class I C, e.g., propafenone, flecainide (acetate); antiarrhythmics of Class II, beta-receptors blockers such as metoprolole, esmolol, propranolol, metoprolol, atenolol, oxprenolol; antiarrhythmics of Class III such as amiodarone, sotalol; antiarrhythmics of Class IV such as diltiazem, verapamil, gallopamil; other antiarrhythmics like adenosine, orciprenaline, ipratropium bromide; agents for stimulation of angiogenesis in the myocardia like Vascular Endothelial Growth Factor (VEGF), Basic Fibroblast Growth Factor (bFGF), non-viral DNA, viral DNA, endothelial growth factors: FGF-1, FGF-2, VEGF, TGF; antibodies, monoclonal antibodies, anticaline; stem cells, Endothelial Progenitor Cells (EPC); digitalisglycosides such as acetyldigoxine/metildigoxine, digitoxin, digoxin; heart glycosides such as quabaine, proscillaridine; antihypertension drugs like central-functioning antiadrenal energy substances, e.g., centrally active antiadrenergic substances, e.g. methyldopa, imidazoline receptor-agonists; calcium channel blockers of the dihydropyridine type such as nifedipine, nitrendipine; ACE-inhibitors: quinaprilate, cilazapril, moexipril, trandolapril, spirapril, imidapril, trandolapril; angiotensin-II-antagonists: candesartane cilexetil, valsartane, telmisartane, olmesartane medoxomil, eprosartane; peripherally operating alpha-Receptor blockers such as prazosin, urapidil, doxazosin, bunazosin, terazosin, indoramine; vaso-dilatators like dihydralazine, diisopropylaminedichloracetate, minoxidil, nitroprussid sodium; other anti-hypertension drugs such as indapamide, co-dergocrine mesilate, dihydroergotoxinmethanesulfonate, cicletanin, bosentane, fludrocortisone; phosphodiesterase inhibitors like milrinone, enoximone and antihypotonics, and adrenergic and dopaminergic substances such as dobutamine, epinephrine, etilefrin, norfenefrin, nor epinephrine, oxilofrin, dopamine, midodrin, pholedrin, ameziniummetil; and partial adrenoceptor agonists such as dihydroergotamine; fibronectine, polylysine, ethylene vinyl acetate, inflammatory cytokines like: TGFβ, PDGF, VEGF, bFGF, TNFα, NGF, GM-CSF, IGF-a, IL-1, IL-8, IL-6, Growth Hormone; as well as adhesive substances such as cyanoacrylates, beryllium, silica; and growth factors such as erythropoietin, hormones such as corticotropine, gonadotropine, somatropin, thyrotrophin, desmopressin, terlipressin, oxytocin, cetrorelix, corticorelin, leuprorelin, triptorelin, gonadorelin, ganirelix, buserelin, nafarelin, goserelin, as well as regulating peptides such as somatostatin, octreotide; bone and cartilage stimulating peptides, Bone Morphogenetic Proteins (BMPs), including recombinant BMP's such as, e.g., recombinant human BMP-2 (rhBMP-2), bisphosphonates (e.g. risedronate, pamidronate, ibandronate, zoledronic acid, clodronin acid, etidronic acid, alendronic acid, tiludronic acid), fluorides such as disodiumfluorophosphate, sodium fluoride; calcitonin, dihydrotachystyrene; growth factors and cytokines like Epidermal Growth Factor (EGF), Platelet-Derived Growth Factor (PDGF), Fibroblast Growth Factors (FGFs), Transforming Growth Factors-b TGFs-b), Transforming Growth Factor-a (TGF-a), Erythropoietin (Epo), Insulin-Like Growth Factor-I (IGF-I), Insulin-Like Growth Factor-II (IGF-II), Interleukin-1 (IL-1), Interleukin-2 (IL-2), Interleukin-6 (IL-6), Interleukin-8 (IL-8), Tumor Necrosis Factor-a (TNF-a), Tumor Necrosis Factor-b (TNF-b), Interferon-g (INF-g), Colony Stimulating Factors (CSFs); monocyte chemotactic protein, fibroblast stimulating factor 1, histamine, fibrin or fibrinogen, endothelin-1, angiotensin II, collagens, bromocriptin, methylsergide, methotrexate, carbon tetrachloride, thioacetamide, and ethanol; also silver (ions), titanium dioxide, antibiotics and anti-infectives such as β-lactam-antibiotics, e.g. β-lactamase-sensitive penicillins such as benzyl penicillins (penicillin G), phenoxymethyl penicillin (penicillin V); β-lactamase-resistant penicillins such as aminopenicillins, e.g. amoxicillin, ampicillin or bacampicillin; acylamino penicillins such as mezlocillin or piperacillin; carboxypenicillins, cephalosporines such as cefazolin, cefuroxim, cefoxitin, cefotiam, cefaclor, cefadroxil, cefalexin, loracarbef, cefixim, cefuroximaxetil, ceftibuten, or cefpodoximproxetil; aztreonam, ertapenem, meropenem; β-lactamase inhibitors such as sulbactam, sultamicillintosilate; tetracyclines such as doxycycline, minocycline, tetracycline, chlortetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides like clindamycin, lincomycin, gyrase inhibitors like fluorochinolone like ciprofloxacin, ofloxacin, moxifloxacin, norfloxacin, gatifloxacin, enoxacin, fleroxacin, levofloxacin; chinolones such as pipemide acid; sulfonamides, trimethoprim, sulfadiazine, sulfalene; glycopeptide antibiotics such as vancomycin, teicoplanin; polypeptide antibiotics including polymyxine, colistin, polymyxin-B, nitroimidazole derivatives such as metronidazol, tinidazol; aminochinolones such as chloroquin, mefloquin, hydroxychloroquin; biguanides such as proguanil; chinin alkaloids and diaminopyrimidine such as pyrimethamine; amphenicoles such as chloramphenicol; rifabutin, dapson, fusidinic acid, fosfomycin, nifuratel, telithromycin, fusafungine, fosfomycine, pentamidindiisethionate, rifampicin, taurolidine, atovaquon, linezolid; virustatics such as acyclovir, gancyclovir, famcyclovir, foscamet, inosine-(dimepranol-4-acetamidobenzoate), valgancyclovir, valacyclovir, cidofovir, brivudine; antiretroviral active substances (nucleoside analogous reverse transcriptase inhibitors and derivatives) such as lamivudine, zalcitabine, didanosine, zidovudine, tenofovir, stavudine, abacavire; non-nucleoside analogs reverse-transcriptase inhibitors: amprenavir, indinavir, saquinavir, lopinavir, ritonavir, nelfinavir; amantadine, ribavirine, zanamivir, oseltamivir and lamivudine, and the like, as well as arbitrary combinations and mixtures thereof.

Moreover therapeutically active substances may be selected from microorganisms, plant or animal cells including human cells or cell cultures and tissues, especially recombinant cells or organized cells or tissues which may be obtained from mammals, including heterologic or autologic cells or tissues, or transfected cells, which can express and release physiological or pharmacologically active substances. Stem cells, primary cells, and progenitor cells of differentiated primary cells or arbitrary mixtures thereof may also be used. Cells or organized cells or tissues may also be used as therapeutic agents, which are not transfixed and/or altered by means of gene technology.

### Multifunctional Agents

In accordance with exemplary embodiments of the present invention, various signal generating agents may be coupled with each other to form bifunctional, trifunctional or multifunctional signal generating agents, which may be assembled from a plurality of functional units that are linked with each other. In this manner it is possible to link different signal generating agents with each other, resulting in complex signal generating agents that combine different signal generating properties in a conjugate. Such conjugated signal generating agents may additionally contain targeting groups or therapeutic active substances, which can be joined as therapeutic groups to the conjugated complex. Therefore, in accordance with exemplary embodiments of the present invention, bifunctional signal generating agents may be used comprising a signal generating agent and a further agent having different signal generating properties, including but not limited to: a paramagnetic agent for signal generation by means of MRI and a coupled fluorescence marker as disclosed, for example, in PCT publicaton WO 04/026344; a paramagnetic and a diamagnetic group coupled in an MRI signal generating agent, such as that described, e.g., in European publication EP 1105162 or in PCT publication WO 00/09170; or dimeric signal generating agents of a super paramagnetic or ferromagnetic and X-ray contrast components such as that described in U.S. Pat. No. 5,346,690; or a paramagnetic and an iodated component composite agent for MRI and X-rays as described, e.g., in U.S. Pat. No. 5,242,683. In accordance with further exemplary embodiments of the present invention, bifunctional signal generating agents may also comprise a signal generating agent and a therapeutically active substance, or a signal generating agent and a targeting group. Examples of signal-producing agents combined with therapeutically active substances are described, for example, in U.S. Pat. Nos. 6,207,133, 6,811,766 and 6,479,033, German Patent Nos. 10151791 and 4035187, Canadian Patent No. 1336164, European Patent No. EP0458079, and PCT publications WO 02/051301, WO 97/05904, WO 04/071536 and WO 04/080483. Examples of combined signal-generating agents with targeting groups are described in, e.g., U.S. Pat. Nos. 6,232,295, 6,652,835 and 6,207,133, Canadian Patent No. CN 1224622, PCT publications WO 99/20312, WO 04/071536, WO 97/36619, WO 03/011115 and WO 04/080483, and others.

Trifunctional signal generating agents that may be used in accordance with exemplary embodiments of the present invention may comprise at least one signal generating component and a further signal generating component or a therapeutically active component or a targeting group, and a still further signal generating component or a therapeutically active agent or a targeting group. Multifunctional signal generating agents may be selected from such a trifunctional signal generating agent having at least one other component which can be chosen arbitrarily. U.S. patent application Ser. No. 08/690,612, e.g., describes how multifunctional or multimeric signal-generating agents may be manufactured.

The bi-, tri-, and multi-functional signal generating agents may be present as covalently or non-covalently bonded macromolecules, as micelles or micro spheres, encapsulated in liposomes or in polymers, or bound covalently in polymers. For covalent bonds, conventional substituents in the form of functional groups may be coupled to the individual components. Such functional groups may include, e.g., amino, carboxyl, oxo or thiol groups. These groups can be linked with each other directly or by means of a linker. Conventional linkers such as homo- or hetero-functional linkers have been described in the literature (see, e.g., Pierce Chemical Company catalogue, technical section on cross-linkers, pages 155-200 (1994)). Linkers that may be used in exemplary embodiments of the present invention include but are not limited to alkyl groups (including substituted alkyl groups and alkyl groups with heteroatom groups), short chain alkyl groups, esters, amides, amines, epoxy groups, nucleic acid, peptide, ethylene glycol, hydroxyl, succinimidyl, maleicidyl, biotin, aldehyde or nitrilotriacetate groups, and their derivatives.

In accordance with further exemplary embodiments of the present invention, mono-, bi-, tri- or multi-functional signal generating agents may be linked non-covalently or partially or completely covalently, they may be encapsulated in micelles wherein the micelles may have a diameter of about 2 nm to 800 nm, preferably from about 5 nm to 200 nm, or more preferably from about 10 nm to 25 nm. The size of the micelles used may be chosen based on the number of hydrophobic and hydrophilic groups, on the molecular weight of the signal generating agents used, and on the aggregation number. In aqueous solutions, branched or unbranched amphiphilic substances present as monomer or oligomer or polymer may be used in order to achieve encapsulation of the signal generating agents. The hydrophobic nucleus of the micelles can contain a multiplicity of hydrophobic groups, preferably between 1 and about 200, according to the desired setting of the micelle size. Signal generating agents and targeting groups of the therapeutic agents may also be present in the micelles and may be partially linked covalently with each other.

Hydrophobic groups may comprise hydrocarbon groups or residues or silicone, including for example polysiloxane chains. Moreover they can be chosen from hydrocarbon-based monomers, oligomers and polymers, or from lipids or phospholipids or any desired combinations thereof.

Glyceryl esters may also be used in further exemplary embodiments of the present invention, including but not limited to phosphatidyl ethanolamine, phosphatidyl cholines, or polyglycolides, polylactides, polymethacrylate, polyvinylbutylether, polystyrene, polycyclopentadienylmethylnorbomene, polyethylenepropylene, polyethylene, polyisobutylene, polysiloxane. Hydrophilic polymers may also be selected for encapsulation in micelles, including polystyrene sulfonic acid, poly-N-alkylvinylpyridinium halides, poly(meth)acrylic acid, polyamino acids, poly-N-vinylpyrrolidone, polyhydroxyethylmethacrylate, polyvinyl ether, polyethylene glycol, polypropylene oxide, polysaccharides such as agarose, dextran, starch, cellulose, amylose, amylopectin, or polyethylene glycol or polyethylene imines of arbitrary molecular weight, and these may be chosen according to the desired micelle property. Further, mixtures of hydrophobic or hydrophilic polymers or lipid-polymer compounds may also be employed. In a further exemplary embodiment, the polymer used is conjugated as a block copolymer, wherein hydrophilic as well as hydrophobic polymers or any desired mixtures thereof can be selected to form 2-, 3- or multi-block copolymers.

Signal generating agents encapsulated in micelles and other functional components may be functionalized further, whereby linkers may be attached at any desired positions of the micelle, preferably to amino, thiol, carboxyl, hydroxyl, succinimidyl, maleimidyl, biotin, aldehyde or nitrilotriacetate groups, to which further molecules or compounds may be chemically bonded covalently or non-covalently. Such further molecules may include, e.g., molecules such as proteins, peptides, amino acids, polypeptides, lipoproteins, glycosaminoglycane, DNA, RNA or similar biomolecules.

In accordance with exemplary embodiments of the present invention, mono-, bi-, tri-, or multi-functional signal-generating agents may be used that are non-covalently or partially or completely covalently linked, and which may further be present in microspheres and liposomes. Microspheres having sizes of less than about 1000 μm may be selected from biocompatible synthetic polymers or copolymers, which may further comprise monomers, dimers or oligomers or other preferred pre-polymeric precursors of the following polymerizable substances: acrylic acid, methacrylic acid, ethyleneimine, crotonic acid, acryl amide, ethylacrylate, methylmethacrylate, 2-hydroxyethylmethacrylate (HEMA), lactonic acid, glycolic acid, [epsilon]-caprolactone, acrolein, cyanoacrylate, bisphenol-A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamide, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium 2-sulfoxyethylmethacrylate, vinyl pyridine, aminoethylmethacrylate, 2-methacryloyloxytrimethylammonium chloride, also polyvinylidene, or polyfunctional cross-linked monomers such as for example N,N′-methylene-bis-acrylamide, ethylene glycol dimethacrylat, 2,2′-(p-phenylenedioxy)-diethyl-dimethacrylate, divinylbenzene, triallylamine or methylene-bis-(4-phenylisocyanate), and the like, or their derivatives, or copolymers including any combinations thereof. Polymers that may be used include polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamide (nylon) and the like or their derivatives, or copolymers or any desired mixtures thereof. Copolymers that may be used include but are not limited to polyvinylidene polyacrylonitrile, polyvinylidene polyacrylonitrile polymethylmethacrylate, or polystyrene polyacrylonitrile and the like, or their derivatives, or any mixtures thereof. Methods for the manufacture of such microspheres are described, for example, in U.S. Pat. Nos. 4,179,546, 3,945,956, 3,293,114, 3,401,475, 3,479,811, 3,488,714, 3,615,972, 4,549,892, 4,108,806, 4,540,629, 4,421,562, 4,420,442, 4,898,734, 4,822,534, 3,732,172, 3,594,326 and 3,015,128, Japan Kokai Tokkyo Koho 62 286534, British Patent No. 1,044,680, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964), and others.

Signal-generating agents, present as mono-, bi-, tri-, or multi-functional agents, may also be linked with polymers. A general overview of methods for doing this is described, e.g., in PCT publication PCT/US95/14621 and in U.S. patent application Ser. No. 08/690,612. Signal-generating agents can, for example, be linked with polymers wherein chemical groups are available that allow a bond to be made from the signal-generating agents to the polymer or polymer mixture selected. Polymers are understood to be compounds which contain at least two or three sub-units that are covalently linked to each other. In exemplary embodiments employing such signal generating agents, at least one part of a monomer sub-unit contains one or a plurality of functional groups that allow covalent bonding to the signal-generating agent. In certain exemplary embodiments, coupling groups may be used to link the monomeric sub-groups with the signal-generating agents. A multiplicity of polymers are suitable for use in such exemplary embodiments, including but not limited to functionalized styrenes, such as amino styrene, functionalized dextrane, polyamino acids (poly-D-amino acids as well as poly-L-amino acids), e.g. polylysine, or polymers which contain lysine or other suitable amino acids. Other useful polyamino acids include polyglutamic acids, polyaspartic acid, copolymers of lysine and glutamine or aspartic acid, or copolymers of lysine with alanine, tyrosine, phenylalanine, serine, tryptophan and/or proline.

The polymers used in certain exemplary embodiments of the present invention may be selected from functionalized or non-functionalized polymers including, e.g., thermosets, thermoplastics, synthetic rubbers, extrudable polymers, injection molding polymers, moldable polymers and the like, or mixtures thereof, and such polymers may additionally be used as components of any composites. Further, additives may be chosen which improve the compatibility of the components used, for example coupling agents such as silanes, surfactants or fillers, including organic or inorganic fillers.

In certain exemplary embodiments the polymer may be selected from polyacrylates such as polymethacrylate, unsaturated polyesters, saturated polyesters, polyolefins (for example polyethylene, polypropylene, polybutylene, and the like), alkyd resins, epoxy-polymers, polyamides, polyimides, polyetherimides, polyamideimides, polyesterimides, polyesteramideimides, polyurethanes, polycarbonates, polystyrenes, polyphenols, polyvinylesters, polysilicones, polyacetals, cellulose acetates, polyvinyl chlorides, polyvinyl acetates, polyvinyl alcohols, polysulfones, polyphenylsulfones, polyethersulfones, polyketones, polyetherketones, polyetheretherketones, polyetherketoneketones, polybenzimidazoles, polybenzoxazoles, polybenzthiazoles, polyfluorocarbons, polyphenylenether, polyarylates, cyanatoester-polymers, copolymers comprising two or more of those polymers named above, and the like.

Polymers that may be used in accordance with exemplary embodiments of the present invention may be acrylics, including monoacrylates, diacrylates, triacrylates, tetraacrylates, pentacrylates, and polyacrylates. Examples of polyacrylates include polyisobomylacrylate, polyisobornylmethacrylate, polyethoxyethoxyethylacrylate, poly-2-carboxyethylacrylate, polyethylhexylacrylate, poly-2-hydroxyethylacrylate, poly-2-phenoxylethylacrylate, poly-2-phenoxyethylmethacrylate, poly-2-ethylbutylmethacrylate, poly-9-anthracenylmethyl methacrylate, poly-4-chlorophenylacrylate, polycyclohexylacrylate, polydicyclopentenyloxyethylacrylate, poly-2-(N,N-diethylamino)ethylmethacrylate, poly-dimethylaminoeopentylacrylate, poly-caprolactone 2-(methacryloxy)ethylester, or polyfurfurylmethacrylate, poly(ethylene glycol)methacrylate, polyacrylic acid and poly(propylene glycol)methacrylate.

Examples of usable diacrylates, from which polyacrylates can be produced, include 2,2-bis(4-methacryloxyphenyl)propane, 1,2-butanedioldiacrylate, 1,4-butanediol-diacrylate, 1,4-butanedioldimethacrylate, 1,4-cyclohexanedioldimethacrylate, 1,10-decanedioldimethacrylate, diethyleneglycoldiacrylate, dipropyleneglycoldiacrylate, dimethylpropanedioldimethacrylate, triethyleneglycoldimethacrylate, tetraethyleneglycoldimethacrylate, 1,6-hexanedioldiacrylate, neopentylglycoldiacrylate, polyethyleneglycoldimethacrylate, tripropyleneglycoldiacrylate, 2,2-bis[4-(2-acryloxyethoxy)phenyl]propane, 2,2-bis[4-(2-hydroxy-3-methacryloxypropoxy)phenyl]propane, bis(2-methacryloxyethyl)N,N-1,9-nonylenebiscarbamate, 1,4-cyclohexanedimethanoldimethacrylate, and diacrylic urethane oligomers.

Examples of triacrylates, which can be used for the manufacture of polyacrylates include tris(2-hydroxyethyl)isocyanuratetrimethacrylate, tris(2-hydroxyethyl)-isocyanuratetriacrylate, trimethylolpropanetrimethacrylate, trimethylolpropanetriacrylate or pentaerythritoltriacrylate. Examples of tetraacrylates that may be used include pentaerythritoltetraacrylate, ditrimethylopropane tetraacrylate, or ethoxylated pentaerythritoltetraacrylate. Pentaacrylates that may be used in embodiments of the present invention include dipentaerythritolpentaacrylate and pentaacrylate-ester.

Polyacrylates may also comprise other aliphatic unsaturated organic compounds such as polyacrylamides and unsaturated polyesters formed from condensation reactions of unsaturated dicarboxylic acids and diols, and vinyl compounds, and additionally compounds with terminal double bonds. Examples of vinyl compounds include N-vinylpyrrolidone, styrene, vinyl-naphthalene or vinylphthalimide. Methacrylamide derivates that may be used in practicing the present invention include N-alkyl or N-alkylene-substituted or unsubstituted (meth)acryl amide, such as acryl amide, methacrylamide, N-methacrylamide, N-methylmethacrylamide, N-ethylacrylamide, N,N-dimethylacrylamide, N,N-dimethylmethacrylamide, N,N-diethylacrylamide, N-ethylmethacrylamide, N-methyl-N-ethylacrylamide, N-isopropylacrylamide, N-n-propylacrylamide, N-isopropylmethacrylamide, N-n-propylmethacrylamide, N-acryloyloylpyrrolidine, N-methacryloylpyrrolidine, N-acryloylpiperidine, N-methacryloylpiperidine, N-acryloylhexahydroazepine, N-acryloylmorpholine, or N-methacryloylmorpholine.

Other polymers that may be used in accordance with exemplary embodiments of the present invention include unsaturated and saturated polyesters, including alkyd resins. The polyesters may contain polymeric chains, and/or a plurality of saturated or aromatic dibasic acids or anhydrides. Epoxy resins, which may be used as monomers, oligomers or polymers, including those which contain one or a plurality of oxiran rings, may have an aliphatic, aromatic or mixed aliphatic-aromatic molecular structure, or may comprise only non-benzoides, thus aliphatic or cycloaliphatic structures with or without substituents like halogens, ester groups, ether groups, sulfonate groups, siloxane groups, nitro groups or phosphate groups or any combinations thereof may be used. Epoxy resins that may be used in exemplary embodiments of the present invention include those of the glycidyl-epoxy type, for example with diglycidylether groups of bisphenol-A, or amino-derivatized epoxy resins, tetraglycidyldiaminodiphenylmethane, triglycidyl-p-aminophenol, triglycidyl-m-aminophenol or triglycidylaminocresol and their isomers, phenol-derivatized epoxy resins such as bisphenol-A-epoxy resins, bisphenol-F-epoxy resins, bisphenol-S-epoxy resins, phenol-novolak epoxy resins, cresol-novolak epoxy resins or resorcinol epoxy resins, or alicyclic epoxy resins. Halogenated epoxy resins may also be used, including glycidylethers of polyhydric phenols, diglycidylethers of bisphenol A, glycidylethers of phenol-formaldehyde Novolak resins and resorcinol-digylcidylethers, as well as other epoxy resins, such as those described, e.g., in U.S. Pat. No. 3,018,262. In accordance with exemplary embodiments of the present invention, the choice of resin is not restricted to the examples mentioned alone; and mixtures of two or a plurality of epoxy resins may also be chosen in addition to mono-epoxy components. Epoxy resins may also include UV-cross-linkable and cycloaliphatic resins.

Polymers that may be used further include polyamides (nylons) such as, for example, aliphatic or aromatic polyamides, nylon-6-(polycaprolactam), nylon 6/6 (polyhexamethyleneadipamide), nylon 6/10, nylon 6/12, nylon 6/T (polyhexamethylene terephthalamide), nylon 7 (polyenanthamide), nylon 8 (polycapryllactam), nylon 9 (polypelargonamide), nylon 10, nylon 11, nylon 12, nylon 55, nylon XD6 (poly meta-xylylene adipamide), nylon 6/I, or polyalanine.

Other polymers which may be employed include polyimides, polyetherimides, polyamideimides, polyesterimides, or polyesteramideimides.

In some exemplary embodiments conductive polymers may be selected such as, e.g., saturated or unsaturated polyparaphenylenevinylene, polyparaphenylene, polyaniline, polythiophene, polyazines, polyfuranes, polypyrroles, polyselenophene, poly-p-phenylenesulfide, or polyacetylene, either as monomers, oligomers or polymers, in any combination or mixtures with other monomers, oligomers or polymers or copolymers of the monomers named above. Such polymers may contain one or a plurality of organic radicals, for example alkyl or aryl radicals or the like, or inorganic radicals, such as silicon or germanium or the like, or any mixtures thereof. Conducting or semiconducting polymers may be used, including those with resistivities between about 10¹² and 10⁵ Ohm-cm. Such polymers may comprise complexed metal salts, and may be more easily formed from polymers which contain nitrogen, oxygen, sulfur, halides, or unsaturated double bonds or triple bonds, or other structures which are suitable for complex formation. For example, suitable polymers may include elastomers like polyurethanes and rubbers, adhesive polymers and plastics. Metal salts that may be used include transition metal halides such as CuCl₂, CuBr₂, CoCl₂, ZnCl₂, NiCl₂, FeCl₂, FeBr₂, FeBr₃, CuI₂, FeCl₃, FeI₃, or FeI₂, as well as salts like Cu(NO₃)₂, metal lactates, metal glutamates, metal succinates, metal tartrates, metal phosphates, metal oxalates, LiBF₄, H₄Fe(CN)₆ and the like.

Biocompatible and/or biodegradable, polymers may be used in still further exemplary embodiments, including but not limited to collagens, albumin, gelatin, hyaluronic acid, starch, cellulose (methylcellulose, hydroxypropylcellulose, hydroxypropylmethylcellulose, carboxymethylcellulose phthalate; casein, dextran, polysaccharides, fibrinogen, poly(D,L-lactide), poly(D,L-lactide-coglycolide), poly(glycolide), poly(hydroxybutylate), poly(alkyl carbonate), poly(orthoesters), polyesters, poly(hydroxyvaleric acid), polydioxanone, poly(ethyleneterephthalate), poly(malic acid), poly(tartronic acid), polyanhydride, polyphosphohazene, poly(amino acids), and all their copolymers or any mixtures thereof.

In certain exemplary embodiments, pH-sensitive polymers may be used, including poly(acrylic acid) and its derivatives, for example homopolymers such as poly(amino carboxylic acid), poly(acrylic acid), poly(methyl acrylic acid) and their copolymers. Polysaccharides such as celluloseacetatephthalate, hydroxypropylmethylcellulosephthalate, hydroxypropylmethylcellulosesuccinate, celluloseacetatetrimellitate and chitosan may also be used.

In certain exemplary embodiments, temperature sensitive polymers may be used, including but not limited to: poly(N-isopropylacrylamide-co-sodium-acrylate-co-n-N-alkylacrylamide), poly(N-methyl-N-n-propylacrylamide), poly(N-methyl-N-isopropylacrylamide), poly(N-N-propylmethacrylamide), poly(N-isopropylacrylamide), poly(N,N-diethylacrylamide), poly(N-isopropylmethacrylamide), poly(N-cyclopropylacrylamide), poly(N-ethylacrylamide), poly(N-ethylmethylacrylamide), poly(N-methyl-N-ethylacrylamide), or poly(N-cyclopropylacrylamide). Other polymers with thermogel characteristics that may be used include hydroxypropylcellulose, methylcellulose, hydroxypropylmethylcellulose, ethylhydroxyethylcellulose, and pluronics like F-127, L-122, L-92, L-81, L-61.

In other exemplary embodiments, polymers may be used for the encapsulation of signal-generating agents, wherein predominantly no. covalent bonding exists between mono-, bi- tri- or multi-functional signal generating agents, or wherein the signal-generating agents linked in the polymers as described above are provided in the form of polymer spheres or suspensions or emulsion particles. The manufacture of such capsules using mini- or micro-emulsion is known in the art. (See, e.g., Australian publication AU 9169501, European publications EP 1205492, 1401878, 1240215 and 1352915, U.S. Pat. No. 6,380,281, Chinese publication CN 1262692T, U.S. Patent Publication No. 2004192838, CA 1336218, Belgian publication BE 949722, and an overview provided in German publication DE 10037656; see also S. Kirsch, K. Landfester, O. Shaffer, M. S. El-Aasser: “Particle morphology of carboxylated poly-(n-butyl acrylate)/(poly(methyl methacrylate) composite latex particles investigated by TEM and NMR” Acta Polymerica 50, 347-362 (1999); K. Landfester, N. Bechthold, S. Förster, M. Antonietti: “Evidence for the preservation of the particle identity in miniemulsion polymerization” Macromol. Rapid Commun. 20, 81-84 (1999); K. Landfester, N. Bechthold, F. Tiarks, M. Antonietti: “Miniemulsion polymerization with cationic and nonionic surfactants: A very efficient use of surfactants for heterophase polymerization” Macromolecules 32, 2679-2683 (1999); K. Landfester, N. Bechthold, F. Tiarks, M. Antonietti: “Formulation and stability mechanisms of polymerizable miniemulsions” Macromolecules 32, 5222-5228 (1999); G. Baskar, K. Landfester, M. Antonietti: “Comb-like polymers with octadecyl side chain and carboxyl functional sites: Scope for efficient use in miniemulsion polymerization” Macromolecules 33, 9228-9232 (2000); N. Bechthold, F. Tiarks, M. Willert, K. Landfester, M. Antonietti: “Miniemulsion polymerization: Applications and new materials” Macromol. Symp. 151, 549-555 (2000); N. Bechthold, K. Landfester: “Kinetics of miniemulsion polymerization as revealed by calorinmetry” Macromolecules 33, 4682-4689 (2000); B. M. Budhlall, K. Landfester, D. Nagy, E. D. Sudol, V. L. Dimonie, D. Sagl, A. Klein, M. S. El-Aasser: “Characterization of partially hydrolyzed poly(vinyl alcohol). I. Sequence distribution via H-1 and C-13-NMR and a reversed-phased gradient elution HPLC technique” Macromol. Symp. 155, 63-84 (2000); D. Columbie, K. Landfester, E. D. Sudol, M. S. ElAasser: “Competitive adsorption of the anionic surfactant Triton X-405 on PS latex particles” Langmuir 16, 7905-7913 (2000); S. Kirsch, A. Pfau, K. Landfester, O. Shaffer, M. S. El-Aasser: “Particle morphology of carboxylated poly-(n-butyl acrylate)/poly(methyl methacrylate) composite latex particles” Macromol. Symp. 151, 413-418 (2000); K. Landfester, F. Tiarks, H.-P. Hentze, M. Antonietti: “Polyaddition in miniemulsions: A new route to polymer dispersions” Macromol. Chem. Phys. 201, 1-5 (2000); K. Landfester: “Recent developments in miniemulsions—Formation and stability mechanisms” Macromol. Symp. 150, 171-178 (2000); K. Landfester, M. Willert, M. Antonietti: “Preparation of polymer particles in non-aqueous direct and inverse miniemulsions” Macromolecules 33, 2370-2376 (2000); K. Landfester, M. Antonietti: “The polymerization of acrylonitrile in minlemulsions: ‘Crumpled latex particles’ or polymer nanocrystals” Macromol. Rapid Comm. 21, 820-824 (2000); B. z. Putlitz, K. Landfester, S. Förster, M. Antonietti: “Vesicle forming, single tail hydrocarbon surfactants with sulfonium-headgroup” Langmuir 16, 3003-3005 (2000); B. Z. Putlitz, H.-P. Hentze, K. Landfester, M. Antonietti: “New cationic surfactants with sulfonium-headgroup” Langmuir 16, 3214-3220 (2000); J. Rottstegge, K. Landfester, M. Wilhelm, C. Heldmann, H. W. Spiess: “Different types of water in film formation process of latex dispersions as detected by solid-state nuclear magnetic resonance spectroscopy” Colloid Polym. Sic. 278, 236-244 (2000); M. Antonietti, K. Landfester: “Single molecule chemistry with polymers and colloids: A way to handle complex reactions and physical processes?” Chem. Phys. Chem. 2, 207-210 (2001); K. Landfester, H.-P. Hentze: “Heterophase polymerization in inverse systems” in Reactions and Synthesis in Surfactant Systems, J. Texter, Ed.; Marcel Dekker, Inc.: New York (2001), pp 471-499; K. Landfester: “Polyreactions in miniemulsions” Macromol. Rapid Comm. 896-936 (2001); K. Landfester: “The generation of nanoparticles in miniemulsion” Adv. Mater. 10, 765-768 (2001); K. Landfester: “Chemie—Rezeptionsgeschichte” in Der Neue Pauly—Enzyklopädie der Antike; J. B. Metzler: Stuttgart; Vol. 15 (2001); B. z. Putlitz, K. Landfester, H. Fischer, M. Antonietti: “The generation of ‘armored latexes’ and hollow inorganic shells made of clay sheets by templating cationic miniemulsions and latexes” Adv. Mater. 13, 500-503 (2001); F. Tiarks, K. Landfester, M. Antonietti: “Preparation of polymeric nanocapsules by miniemulsion polymerization” Langmuir 17, 908-917 (2001); F. Tiarks, K. Landfester, M. Antonietti: “Encapsulation of carbon black by miniemulsion polymerization” Macromol. Chem. Phys. 202, 51-60 (2001); F. Tiarks, K. Landfester, M. Antonietti: “One-step preparation of polyurethane dispersions by miniemulsion polyaddition” J. Polym. Sci., Polym. Chem. Ed. 39, 2520-2524 (2001); F. Tiarks, K. Landfester, M. Antonietti: “Silica nanoparticles as surfactants and fillers for latexes made by miniemulsion polymerization” Langimuir 17, 5775-5780 (2001)).

### Materials/Components

Certain exemplary embodiments of the present invention include implantable medical devices or materials for implantable medical devices or their components. In such exemplary embodiments, a bulk material with signal-generating properties may be provided in which the signal generating agents are bound into the material matrix of the implantable medical device. Alternatively, the prepared medical device may be provided, at least in part, with a signal-generating coating. In accordance with exemplary embodiments of the present invention, it is also possible to combine both variants, i.e., a signal generating bulk material and a signal-generating coating.

In one exemplary embodiment, the medical device itself is part of the inventive combination, and the device is combined with at least one signal-generating agent and at least one therapeutically active agent. The signal-generating agent(s) and the therapeutically active agent(s) may be incorporated into the material of the implantable device itself, particularly if the device is made of resorbable or degradable materials. In another exemplary embodiment, the implantable device is itself not part of the inventive combination, and instead it may be, for example, coated with a coating comprising the inventive combination, i.e. the coating comprises at least one signal-generating device, at least one therapeutically active agent and at least one material for the manufacture of an implantable medical device. The coating may comprise a suitable coating material such as pyrolytic carbon, a polymer, a film coating, or the like.

The term “at least one material for the preparation of an implantable medical device and/or at least one component of an implantable medical device” includes all of the above-described exemplary embodiments.

In accordance with exemplary embodiments of the present invention, the implantable medical device or component of the implantable medical device provided may comprise a planar or spherical body, or any desired three-dimensional shape in different dimensions, including tubular or other hollow body shapes. The shape of the implantable medical device or component of the implantable medical device. will generally not limit application of the exemplary embodiments of the present invention.

Implantable medical devices include any devices that are designated to be incorporated into an organism as ultra short term, short term, or long term devices, and which may be used for diagnostic, therapeutic or prophylactic purposes, or for combined diagnostic/therapeutic/prophylactic purposes. The terms “implantable medical device” and “implant” are used herein synonymously. In accordance with the invention the selected organisms in certain embodiments are mammals. Mammals in accordance with the invention include all mammals, including but not limited to domestic animals such as dogs and cats, agricultural livestock such as cattle, sheep or goats, laboratory animals such as mice or rats, primates such as apes, chimpanzees,and the like, and humans. In some embodiments, implants and implanted active substances may be selected which are designated for utilization in humans.

The implantable medical devices used in certain exemplary embodiments of the present invention are not limited to any particular implant type and may include, for example, vessel endoprostheses, intraluminal endoprotheses, stents, coronary stents, peripheral stents, pacemakers or parts thereof, surgical and orthopedic implants for temporary purposes such as joint socket inserts, surgical screws, plates, nails, implantable orthopedic supporting aids, surgical and orthopedic implants such as bones or joint prostheses, for example artificial hip or knee joints, bone and body vertebra means, artificial hearts or parts thereof, artificial heart valves, cardiac pacemaker housings, electrodes, subcutaneous and/or intramuscular implants, active substance repositories, or microchips or the like. Materials for implantable medical devices may be selected from non-degradable or completely degradable materials or any combinations thereof. Implant materials may also consist entirely of metal-based materials or alloys or composites, or laminated materials, carbon, or carbon composites, as well as composite materials of these named materials, or any desired combinations thereof.

In certain exemplary embodiments, ceramic and/or metal-based materials may be used, including for example amorphous and/or (partly) crystalline carbon, massive carbon material (“Vollkarbon”), porous carbon, graphite, carbon composite materials, carbon fibers; ceramics including e.g. zeolites, silicates, aluminum oxides and aluminum silicates; silicon carbide or silicon nitride; metal carbides, metal oxides, metal nitrides, metal carbonitrides, metal oxycarbides, metal oxynitrides and metal oxycarbonitrides of the transition metals such as titanium, zirconium, hafnium, vanadium, niobium, tantalum, chromium, molybdenum, tungsten, manganese, rhenium, iron, cobalt, nickel; metals and metal alloys, including the noble metals gold, silver, ruthenium, rhodium, palladium, osmium, iridium, platinum, and their alloys; metals and metal alloys of titanium, zirconium, hafnium, vanadium, niobium, tantalum, chromium, molybdenum, tungsten, manganese, rhenium, iron, cobalt, nickel, copper, magnesium; steel, including stainless steel, such as but not limited to Fe-18Cr-14Ni-2.5Mo (“316LVM” ASTM F138), Fe-21Cr-10Ni-3.5Mn-2.5Mo (ASTM F 1586), Fe-22Cr-13Ni-5Mn (ASTM F 1314), Fe-23Mn-21Cr-1Mo-1N (nickel-free stainless steel), or platinum-containing radiopaque steel alloys, so called PERSS (platinum enhanced radiopaque stainless steel alloys), as well as shape memory alloys such as, e.g., nitinol and other nickel-titanium alloys; glass, stone, glass fibers, minerals; natural or synthetic bone substance; imitation bone based on alkaline earth carbonates such as calcium carbonate magnesium carbonate, strontium carbonate, or hydroxyapatite; as well as any combinations of the materials mentioned herein.

In further exemplary embodiments, polymers that may be used include polyacrylates such as polymethyl methacrylates, or those made from unsaturated polyesters, saturated polyesters, a polyolefin (for example polyethylene, polypropylene, polybutylene, and the like), an alkyd resin, an epoxy-polymer, a polyamide, a polyimide, polyetherimide, a polyamideimide, a polyesterimide, a polyesteramideimide, polyurethane, polycarbonate, polystyrene, polyphenol, polyvinylester, polysilicone, polyacetal, celluloseacetate, polyvinyl chloride, polyvinyl acetate, polyvinyl alcohols, polysulfones, polyphenylsulfones, polyethersulfones, polyketones, polyetherketones, polyetheretherketones, polyetherketoneketones, polybenzimidazoles, polybenzoxazoles, polybenzthiazoles, polyfluorocarbons, polyphenyleneethers, polyarylates, cyanatoester-polymers, copolymers of two or more of those polymers noted above, and the like.

Acrylics may also be used, including monoacrylates, diacrylates, triacrylates, tetraacrylates, pentacrylates, and the like The polyacrylates include such compositions as polyisobomylacrylate, polyisobornylmethacrylates, polyethoxyethoxyethylacrylates, poly-2-carboxyethylacrylates, polyethylhexylacrylates, poly-2-hydroxyethylacrylates, poly-2-phenoxylethylacrylates, poly-2-phenoxyethylmethacrylates, poly-2-ethylbutylmethacrylates, poly-9-anthracenylmethyl methacrylates, poly-4-chlorophenylacrylates, polycyclohexylacrylates, polydicyclopentenyloxyethylacrylates, poly-2-(N,N-diethylamino)ethylmethacrylates, poly-dimethylaminoeopentylacrylates, poly-caprolactone 2-(methacryloxy)ethyl esters, or polyfurfurylmethacrylates, poly(ethylene glycol)methacrylates, polyacrylic acid and poly(propylene glycol)methacrylates.

Examples of diacrylates that may be used in certain embodiments, and from which polyacrylates can be manufactured, are 2,2-bis(4-methacryloxyphenyl)propane, 1,2-butanedioldiacrylate, 1,4-butanedioldiacrylate, 1,4-butanedioldimethacrylate, 1,4-cyclohexanedioldimethacrylate, 1,10-decanedioldimethacrylate, diethyleneglycoldiacrylate, dipropyleneglycoldiacrylate, dimethylpropanedioldimethacrylate, triethyleneglycol-dimethacrylate, tetraethyleneglycoldimethacrylate, 1,6-hexanedioldiacrylate, neopentylglycoldiacrylate, polyethyleneglycoldimethacrylate, tripropyleneglycoldiacrylate, 2,2-bis[4-(2-acryloxyethoxy)phenyl]propane,2,2-bis[4-(2-hydroxy-3-ethacryloxypropoxy)-phenyl]propane, bis(2-methacryloxyethyl)N,N-1,9-nonylene-biscarbamate, 1,4-cyclohexane-dimethanoldimethacrylate, and diacrylic urethane oligomers.

Examples of triacrylates that may be used to make polyacrylates in accordance with exemplary embodiments of the present invention include tris(2-hydroxyethyl)isocyanuratetrimethacrylate, tris(2-hydroxyethyl)isocyanuratetriacrylate, trimethylolpropanetrimethacrylate, trimethylolpropanetriacrylate or pentaerythritol-triacrylate. Examples of tetraacrylates that may be used include pentaerythritoltetraacrylate, ditrimethylopropane tetraacrylate, or ethoxylated pentaerythritoltetraacrylate. Examples of pentaacrylates that may be used include dipentaerythritolpentaacrylate and pentaacrylate esters.

Polyacrylates may also comprise other unsaturated aliphatic organic compounds such as, e.g., polyacrylamides and unsaturated polyesters from condensation reactions of unsaturated dicarboxylic acids and diols, and vinyl compounds, and also compounds having terminal double bonds. Examples of vinyl compounds that may be used include N-vinylpyrrolidone, styrene, vinyl naphthalene or vinylphthalimide. Methacrylamide derivates may also be used and may be N-alkyl- or N-alkylene-substituted or unsubstituted (meth)acryl amide, including but not limited to acryl amide, methacrylamide, N-methacrylamide, N-methylmethacrylamide, N-ethylacrylamide, N,N-dimethylacrylamide, N,N-dimethylmethacrylamide, N,N-diethylacrylamide, N-ethylmethacrylamide, N-methyl-N-ethylacrylamide, N-isopropylacrylamide, N-n-propylacrylamide, N-isopropylmethacrylamide, N-n-propylmethacrylamide, N-acryloyloylpyrrolidine, N-methacryloylpyrrolidine, N-acryloylpiperidine, N-methacryloylpiperidine, N-acryloylhexahydroazepine, N-acryloylmorpholine, or N-methacryloylmorpholine.

Other polymers that may be used in accordance with exemplary embodiments of the present invention include unsaturated and saturated polyesters, and alkyd resins. The polyesters may contain polymer chains comprising a various number of saturated or aromatic dibasic acids and anhydrides. Other epoxy resins that can be used may comprise monomers, oligomers or polymers which may contain one or a plurality of oxiran rings, which may further have an aliphatic, aromatic or mixed aliphatic-aromatic molecular structure, or which may be exclusively non-benzenoids, and therefore may be aliphatic or cycloaliphatic, and they may comprise structures with or without substituents such as halogens, ester groups, ether groups, sulfonate groups, siloxane groups, nitro groups or phosphate groups, or any combinations thereof. Epoxy resins that may be used also include the glycidyl-epoxy type, for example those having diglycidylether groups of bisphenol-A. Amino derivatized epoxy resins may also be used including but not limited to tetraglycidyldiaminodiphenylmethane, triglycidyl-p-aminophenol, triglycidyl-m-aminophenol or triglycidylaminocresol and their isomers, phenol derivatized epoxy resins such as bisphenol-A epoxy resins, bisphenol-F epoxy resins, bisphenol-S epoxy-resins, phenol-novolak-epoxy resins, cresol-novolak-epoxy resins or resorcinol epoxy resins, or alicyclic epoxy resins. Halogenated epoxy resins may also be used in accordance with embodiments of the present invention including, e.g., glycidylether of polyhydric phenols, diglycidylether of bisphenol A, glycidylethers of phenol-formaldehyde novolak resins and resorcinol-digylcidylether, as well as other epoxy resins such as those described in U.S. Pat. No. 3,018,262. In accordance with exemplary embodiments of the present invention, mixtures of two or three or more of the named epoxy resins may be used, as well also as mono-epoxy components. The epoxy resins that may be used include UV-cross-linked and cycloaliphatic resins.

Polymers that may be used further include polyamides, such as aliphatic or aromatic polyamides, or Nylon-6-(polycaprolactam), nylon 6/6 (polyhexamethyleneadipamide), nylon 6/10, nylon 6/12, nylon 6T (polyhexamethylene terephthalamide), nylon 7 (polyenanthamide), nylon 8 (polycapryllactam), nylon 9 (polypelargonamide), nylon 10, nylon 11, nylon 12, nylon 55, nylon XD6 (poly meta-xylylene adipamide), nylon 6/I, or poly-alanine.

Other polymers which may be employed include polyimides, polyetherimides, polyamideimides, polyesterimides, and polyesteramideirnides.

In some exemplary embodiments, conductive polymers may be selected such as, e.g., saturated or unsaturated polyparaphenylenevinylene, polyparaphenylene, polyaniline, polythiophene, polyazines, polyfuranes, polypyrroles, polyselenophene, poly-p-phenylenesulfide, or polyacetylene, either as monomers, oligomers or polymers, in any combination or mixtures with other monomers, oligomers or polymers or copolymers of the monomers named above. Such polymers may contain one or a plurality of organic radicals, for example alkyl or aryl radicals or the like, or inorganic radicals, such as silicon or germanium or the like, or any mixtures thereof. Conducting or semiconducting polymers may be used, including those with resistivities between about 10¹² and 10⁵ Ohm-cm. Such polymers may comprise complexed metal salts, and may be more easily formed from polymers which contain nitrogen, oxygen, sulfur, halides, or unsaturated double bonds or triple bonds, or other structures which are suitable for complex formation. For example, suitable polymers may include elastomers like polyurethanes and rubbers, adhesive polymers and plastics. Metal salts that may be used include transition metal halides such as CuCl₂, CuBr₂, CoCl₂, ZnCl₂, NiCl₂, FeCl₂, FeBr₂, FeBr₃, CuI₂, FeCl₃, FeI₃, or FeI₂, as well as salts like Cu(NO₃)₂, metal lactates, metal glutamates, metal succinates, metal tartrates, metal phosphates, metal oxalates, LiBF₄, H₄Fe(CN)₆ and the like.

Biocompatible and/or biodegradable polymers may be used in still further exemplary embodiments, including but not limited to collagens, albumin, gelatin, hyaluronic acid, starch, cellulose (methylcellulose, hydroxypropylcellulose, hydroxypropylmethylcellulose, carboxymethylcellulose phthalate; casein, dextran, polysaccharides, fibrinogen, poly(D,L-lactide), poly(D,L-lactide-coglycolide), poly(glycolide), poly(hydroxybutylate), poly(alkyl carbonate), poly(orthoesters), polyesters, poly(hydroxyvaleric acid), polydioxanone, poly(ethyleneterephthalate), poly(malic acid), poly(tartronic acid), polyanhydride, polyphosphohazene, poly(amino acids), and all their copolymers or any mixtures thereof.

Degradable materials that may be used further include metal-based materials such as, e.g., biodegradable or biocorrodible metal alloys, including but not limited to magnesium alloys, or degradable glass-ceramic materials such as bioglass, silicates, or ceramic or ceramic-type materials such as hydroxyapatite and the like.

Implantable medical devices that are in accordance with exemplary embodiments of the present invention may be non-degradable, or partly degradable, or completely biodegradable devices, including but not limited to implants for complete or partial bone replacement, implants for complete or partial joint replacement, implants for complete or partial vessel replacement, coronary or peripheral stents, or other endoluminal vessel implants, which may be used for complete or partial vessel replacement and/or as active agent repositories or seed implants.

### Material Choice

The selection of the individual elements used in various exemplary embodiments of the present invention has a special importance. In the manufacture or use of signal-generating materials and the selection of the implants or implant materials, the primary medical indication and the desired signal generating modalities should be considered. Such selections may be made in accordance with the invention using the exemplary, but not limiting, guidelines that follow.

Factors to be considered in determining the choice of signal generating agents to be used in exemplary embodiments of the present invention may include one or more of the following:
a) Whether the signal generating agents are to be selected exclusively for marking the implantable medical device;
b) Whether the signal generating agents are to be selected exclusively for marking of surrounding tissue or of compartments at the immediate or communicable boundary area of the implantable medical device;
c) Whether the signal-generating agents are to be chosen exclusively for marking of any specific tissues, cell types, organs or organ regions away from the boundary area of the implantable medical devices, wherein such implantable medical devices may have the primary purpose of introducing signal-generating agents into the organism;
d) Whether the signal-generating agents, in addition to marking of the implant, are also to be selected for marking the surrounding tissue or compartments in the immediate or communicable boundary area of the implant;
e) Whether the signal-generating agents, in addition to marking the implantable medical device, are also to be selected for marking any desired independent tissues, cell types, organs or organ regions away from the boundary area of the implantable medical device, wherein such devices may have the primary purpose of introducing signal-generating agents into the organism;
f) Whether the signal generating agents are chosen primarily for the marking of surrounding tissues, or of compartments in the immediate or communicable boundary area to the implantable medical device, rather than for marking of the implantable medical device itself;
g) Whether the signal-generating agents are chosen primarily for marking of tissues, cell types, organs or organ regions that may lie away from the boundary area of the device, rather than or also for marking of the implantable medical device itself, and wherein such devices may further have the exclusive purpose of introducing signal-generating agents into the organism;
h) Whether the signal generating agents are selected primarily for marking of surrounding tissues, or of compartments in the immediate or communicable boundary area of the device and/or for marking of any cell types, organs or organ regions that may lie away from the device boundary area, rather than or also for marking of the implantable medical device itself, wherein the device may further have the primary purpose of introducing signal-generating agents into the organism;
i) Whether signal-generating agents are also to be combined with therapeutic agents, and either or both types of agents are subject to any of the factors presented in (a) through (h) above;
j) Whether more than one signal-generating agent is chosen to produce combined signal-generating agents having different signal modalities, wherein the modalities may include the physical and chemical properties of an agent and the detection methods that may be used to detect the agent; and
k) Whether signal-generating agents selected according to any of the factors presented in (a) through (j) above are direct, indirect, or mixed signal-generating agents.

Additional factors that may be considered in determining the choice of signal generating agents relate to the desired or needed duration for detection of the signal-generating agents, including:
a) Whether signal-generating agents should be verifiable for ultra-short periods, where ‘ultra short periods’ is understood to mean detection periods ranging from a few seconds or less up to about 3 days;
b) Whether signal generating agents should be verifiable for short periods, where ‘short periods’ is understood to mean detection periods ranging frorm about 3 days to about 3 months.
c) Whether signal-generating agents should be long term verifiable, where ‘long term verifiable’ is understood to mean detectable for periods that range about 3 months to about 12 months;
d) Whether signal generating agents are to be permanently verifiable, where ‘permanently verifiable’ is understood to correspond to detection periods of at least 12 months or longer, and preferably for the total lifetime of a non-degradable implant.

Additional factors that may influence the choice of of signal generating agents relate to the modalities desired. Among these factors are:
a) Which modality is preferred for the desired detection method, e.g. radiographic modalities for X-ray, MRI, and fluorescence based detection methods;
b) Which modalities may be useful when combined, for example the combination of radiopaque and paramagnetic signal-generating agents; and
c) Which modalities are appropriate for combining with chosen therapeutic signal-generating agents.

Other factors that may also influence the choice of signal-generating agents relate to the desired functionality of both the signal-generating agents and the underlying implantable medical device. These factors include but are not limited to:
a) Whether signal-generating agents are chosen exclusively or primarily for verification of the correct anatomical location of the medical device;
b) Whether signal-generating agents are to be used to assist in control of the operation of the implantable medical device, for example, to detect the extent of degradation in a biodegradable implant;
c) Whether the signal-generating agents are used exclusively or primarily to detect the interaction of the implantable medical device with the bordering tissues including, e.g. for detecting engraftment and/or inflammatory reactions in the immediate or communicable surroundings of an implant;
d) Whether signal-generating agents are to be used exclusively or primarily to help control the release of additives, including use in so-called combined implantable medical devices with drug-delivery function, e.g. drug-eluting stents and the like, where such devices may further comprise both signal-generating agents and therapeutic agents; and
e) Whether signal-generating agents are to satisfy more than one of the functions identified in the factors presented in (a) through (d) immediately above.

In accordance with exemplary embodiments of the present invention, the underlying material or the composition or combination of implantable medical devices or of components of implantable medical devices, each may comprise non-degradable or partially degradable or completely degradable materials. The choice of the composition or combination may be based primarily on the purpose and intended function(s) of the signal-generating agents, or conversely the signal-generating agents may be chosen based primarily on the selected material of an implantable medical device. It is known that the material choice is generally made in consideration of both the intended use and the purpose of providing signal-generating agents to the implantable medical device, which in turn relate to the underlying primary illness.

Additional factors may be considered when choosing implant materials for complete or partial introduction of signal-generating agents into the integrated material system in accordance with exemplary embodiments of the present invention. Such factors include:
a) Whether the material is to be manufactured by means of thermal sintering processes, wherein the integration of signal-generating material into the implant matrix may be carried out before or during the manufacture;
b) Whether the material is to be manufactured by means of thermal sintering processes, wherein the integration of the signal-generating material into the implant matrix is carried out after the manufacture, and further wherein at least one open-pore material layer is present;
c) Whether the material is to be manufactured using chemical processes in the absence of thermal stress, which may lead to a degradation or partial degradation of signal-generating materials in the provided form, wherein the integration of signal-generating material into the implant material is carried out before or during the manufacture;
d) Whether the material is to be manufactured by means of chemical processes in the absence of thermal stress, which may lead to a degradation or partial degradation of signal-generating materials in the implant material, wherein the integration of signal-generating materials into the implant matrix is carried out after the manufacture, and further wherein at least one open-pore material layer must be present; and
e) Whether the material is to be completely, partially, or non-degradable in addition to one or more of the factors presented in (a) through (d) immediately above.

Factors to be considered for exemplary embodiments of the present invention wherein complete or partial incorporation of signal-generating agents is provided in the form of a coating include:
a) Whether the coating is to be manufactured by means of thermal sintering processes, plasma spraying, sputtering methods, etc, wherein the integration of signal-generating materials into the coating may be carried out before or during the manufacture;
b) Whether the coating is to be manufactured by means of thermal sintering processes, plasma spraying, sputtering methods, etc, wherein the integration of signal-generating materials into the coating may be carried out after the manufacture, and further wherein the coating may be either closed or porous;
c) Whether the coating is to be manufactured by means of chemical or thermal processes, which may lead to a degradation or partial degradation of signal-generating agents in the provided form, wherein the integration of signal-generating material into the coating may be carried out before or during the manufacture;
d) Whether the coating is manufactured by means of chemical processes in the absence of thermal treatment, which may lead to a degradation or partial degradation of signal-generating agents in the provided form, wherein the integration of signal-generating material into the coating may be carried out after the manufacture;
e) Whether the material is to be completely, partially, or non-degradable in addition to one or more of the factors presented in (a) through (d) immediately above.

For exemplary embodiments of the present invention that comprise non-porous and non-degradable implants, the signal-generating agents may preferably be introduced through a coating of the implant. The coating may be selected from degradable or non-degradable materials, wherein the incorporation of signal-generating agents and/or therapeutically active agents can be carried out during or after manufacture. Applying the coating to the implant may be achieved by any desired coating method, including those that may be known in the art. Thermal coating methods necessitate the use of thermally stable signal-generating agents. Non-thermal methods such as spray-coating, dip-coating, etc. may allow a broader choice of signal-producing agents and combinations thereof. If degradable coatings are chosen, then biodegradable types of coatings may be used including, for example, those formulated with polymers as mixtures, wherein the signal-generating agents are provided from solutions, suspensions, emulsions, dispersions, powders or the like, or those formulated with signal-generating agents covalently linked to polymer formulations. Degradable coatings having bi-functional, tri-functional or multi-functional signal-generating agents may also be used, and further may be combined with at least one therapeutic agent.

In a further exemplary embodiment, the implantable device or a part thereof comprises a porous material, e.g. a porous coating on the device, which may be degradable or not, wherein signal-generating agents are incorporated into the material, e.g. as a reticulated network of particles. In this exemplary embodiment it is preferred to select at least one therapeutically active agent that can be soaked or adsorbed or absorbed into the porous material by techniques known in the art, e.g. by the use of appropriate drug solvents whereby the device or coating may be dipped into or sprayed with a therapeutically active agent-containing solution, with subsequent incorporation of the drug into the porous material.

In certain exemplary embodiments signal-generating agents may be provided in porous inorganic, organic or inorganic-organic coatings, including those comprising composite materials. Such porous coatings may comprise ceramic or metal-based materials. They may also be biodegradable, for example they may comprise hydroxylapatites or analogs or derivatives or similar, or degradable bioglass species. These inherently signal-generating materials may be integrated with other signal-generating agents, either of the same modality for the strengthening of the image-forming signal, or one or a plurality of other modalities; including other signal-generating agents that may comprise nanoparticles. Biocompatible signal-generating agents are generally preferred for use with degradable coatings. Porous coatings may be provided from signal-generating agents, including but not limited to non-degradable or degradable inorganic or organic or mixed inorganic-organic composites, which further may be formed from polymers, nano- or micro-morphous precursors, or from metal-based nanoparticles. Degradable implants may be provided with degradable signal-generating coatings having the same or similar or shorter degradation times as the implants. For exemplary embodiments in which the signal generation is provided primarily to indicate correct anatomical location or is semiquantitatively related to the course and therapy control of the degradation, of engraftment, and/or the interaction with the surrounding tissues, it may be provided through a coating on non-porous degradable implants. Further, a coating of non-porous and degradable implants may be used when its material leads to an impairment of the implant function relative to the material properties if signal-generating agents are incorporated into the material composite. For example, with biodegradable implants such as stents, which may comprise biodegradable polymers such as PLA, mechanical stability for an implant function may not be provided if foreign substances such as pharmacologically active substances are employed. Signal generating coatings for degradable implants may be provided in the form of coatings, in which signal-generating agents are provided in forms such as, e.g., biocompatible nanoparticles, liposomes, micelles, microspheres, and the like, and which further may be embedded in degradable polymers. In some exemplary embodiments, coatings may have radiopaque signaling properties, they may comprise bi-, tri- or multi-functional signal-generating agents, andor they may further comprise therapeutic agents.

In one exemplary embodiment, implants are prepared from biocompatible, essentially non-toxic metal alloys, including but not limited to magnesium or zinc-based alloys, which may be degraded by means of corrosion. If therapeutically active substances are released from the materials during the decomposition of such implants in the body, one may optionally, in accordance with exemplary embodiments of the present invention, do without the addition of a separate active ingredient.

Thus, in some exemplary embodiments, a magnesium or zinc alloy-based implant or part of an implant, (such as a stent) may be used, wherein the alloy comprises the therapeutically active agents. For example, magnesium ions may be liberated by and during degradation upon exposure to bodily fluids in human or animal organisms, resulting in the physiologically induced formation of H2, hydroxyl apatite, and magnesium ions. In these exemplary embodiments, the release and availability of magnesium ions and the formation of hydroxyl apatite have biological effects that are known in the art.

The implant itself or a part thereof may comprise magnesium and/or zinc in the implant material itself, or alternatively in a coating. For example an implant may be partially or fully coated with magnesium and/or zinc particles embedded in a polymeric matrix or in another coating material. In these exemplary embodiments, the combination of therapeutically active and signalling agent with the implant material may be achieved by the use of the signalling agent, Mg or Zn, as a component of the alloy itself or as a part of the implant, or as a part of a coating.

Such implants may further be provided with biodegradable signal-generating coatings, wherein signal-generating agents may be provided directly or alternatively incorporated into degradable polymers as nanoparticles, in the form of liposomes, microspheres, macrospheres, encapsulated in micelles or polymers, or bonded covalently to polymers. Such agents may be bi-, tri-, or multi-functional signal-generating agents, and may further be provided together with at least one therapeutic agent. Such implants may also be provided with biodegradable porous coatings, for example coatings comprising hydroxylapatite and derivatives or analogs thereof, or bioglass. In these exemplary embodiments, biocompatible or biodegradable signal-generating agents comprising nanomorphous particles may be incorporated into the porous coatings, or any desired form of biocompatible or biodegradable signal-generating agents, or both combined, may be incorporated into the hollow spaces of the porous matrix. Degradable porous coatings comprising signal-generating agents having the initial form of nanomorphous particles may be provided, wherein the hollow spaces of such signal generating porous coatings may be charged additionally with other signal-generating agents having any form. Further, non-porous degradable coatings of signal-generating agents may be provided, including those comprising degradable nanomorphous particles.

In accordance with other exemplary embodiments of the present invention, signal-generating agents may be incorporated as precursor components of the implant material from which essentially non-porous or essentially non-degradable implants are formed. Thermally stable forms of the signal-generating agents are preferred if thermal methods or processing steps are used to manufacture the implant. For metal-based implant materials, signal-generating agents may be used which impart to the inherent signal-generating properties of the starting material used at least one other additional signal-generating property. Essentially non-porous and non-degradable implants may comprise polymer materials or polymer composite materials, wherein signal-generating agents may be added to the reactant components used to form the polymer material in the form of solutions, emulsions, suspensions, dispersions, powders and the like, or in the form of covalent components derived from monomers, dimers, trimers or oligomers, or alternatively in the form of prepolymeric precursors which can be synthesized to form polymers, and the polymeric material(s) may be produced therefrom. Essentially non-porous and non-degradable implants comprising polymeric materials or polymer composite materials may be provided with at least one modality representing a signal-generating property, or alternatively with bi-functional, tri-functional or multi-functional signal-generating agents, wherein the non-porous and non-degradable materials or implants in select embodiments do not contain any therapeutic agents or targeting groups within the materials.

The reactant components used to form the implant material in non-porous and non-degradable implants may be provided with signal-generating agents in a suitably finished form, and the finished implant may further be provided with an additional signal-generating coating.

Signal-generating agents may be added as a part of the precursor components to the implant material of essentially non-porous and essentially degradable implants. Implant materials used in accordance with embodiments of the present invention include polymers or polymer composites as well as degradable metal-based materials or their degradable composites, or alternatively, materials based on naturally occurring apatites, hydroxylapatites, their analogs and derivatives thereof, or materials comparable to bone substitute or based on bioglass species. Signal-generating agents to be used with essentially non-porous and essentially degradable implants comprising polymer materials or polymer composite materials may be added to the reactant components from solutions, emulsions, suspensions, dispersions, powders and the like, or added as covalent components of monomers, dimers, trimers or oligomers or other pre-polymer precursors, which may be further synthesized to polymers to produce the active substance therefrom. In contrast to the compositions described in PCT publication WO 04/064611, signal-generating agents may be added to biodegradable polymers such as, e.g., polylactides, polyglycolides, their derivatives and mixtures thereof or their copolymers, wherein the signal generating agents may have radiopaque properties and when combined may have at least one other modality, including e.g. bifunctional radiopaque properties in combination with a therapeutic agent or at least one non-radiopaque modality. Signal-generating agents may be coupled with one or a plurality of targeting groups and/or a plurality of therapeutic agents. Such agents may be combined with materials which are based on naturally occurring apatites, hydroxyl apatites, their analogs and derivatives, comparable bone substitutes or bioglass and the like. Signal-generating agents may be added to the reactant components of non-porous and degradable implants in a suitable form to provide the shaped implant with an additional signal-generating coating.

In one exemplary embodiment, implants may be prepared from biocompatible, essentially non-toxic, metal alloys, which may be degraded by means of corrosion, including but not limited to magnesium- or zinc-based alloys. Thermally stable finished forms of signal-generating agents may be added to the components of such implant materials if these materials are manufactured using conventional thermal methods. Signal-generating agents used in this embodiment may have radiopaque properties, or they may be bi- or tri-functional or multi-functional signal-generating agents provided in suitable forms, and they may further be coupled with therapeutic agents and/or targeting groups.

Signal-generating agents may be added to the reactant components of nonporous and degradable implant materials in a suitable form, so as to provide the formed implant with an additional signal-generating coating.

Porous, essentially non-degradable or degradable implants may already contain signal-generating agents in their material composite structure, such as in the embodiments described above. Porous implants may also be provided with signal-generating agents after their manufacture. Certain exemplary embodiments of the present invention may include implants comprising a porous composite material resulting from the manufacturing process, or alternatively, implants may be provided with porous coatings. Implants may have a porous material structure with average pore sizes ranging from about 1 nm to 10 nm, or preferably from about 1 nm to 10 μm, or more preferably from about 2 nm to 1 μm. It may be important in some embodiments to provide at least one sufficiently porous surface which can be loaded with signal-generating agents, wherein this surface may be created later in the implant manufacturing process or not, and further wherein the porosity may be produced by a specific implant manufacturing process or provided by an open-pore material composite.

The signal generating agents may be introduced into the porous compartments from solutions, suspensions, dispersions or emulsions, or further through the use of additives such as surfactants, stabilizers, flow improvers and the like, by means of suitable methods such as dipping, spraying, injection methods or other appropriate methods.

Porous implants may comprise materials including but not limited to polymers, glasses, metals, alloys, bone, stone, ceramics, minerals or composites. These materials may be degradable, non-degradable, or partially degradable. Provided signal-generating agents may be monofunctional, or alternatively they may be bi-functional or tri-functional, and may further be coupled with therapeutic agents.

In other exemplary embodiments, porous materials may be produced with introduction of appropriate forms of signal generating agents. Thus non-degradable polymers, polymer composites or ceramics or ceramic composites or metal-based materials or metal-based composites or similar materials may already contain signal-generating materials in the form of fillers introduced during the manufacturing process, so that they serve as components of the basic material matrix of the overall composition. Signal-generating agents may further be encapsulated in polymers, for example in the form of polymer capsules, drops or beads, and may be produced through the use of mini- or micro-emulsions. Signal-generating agents for polymer-based materials may be encapsulated in polymers, micelles, liposomes or microspheres, or they may be present as nanoparticles or as components thereof. Implantable medical devices used in accordance with exemplary embodiments of the present invention may have a porous matrix structure in-vivo arising from the the basic material matrix that may remain after fillers and signal-generating agents and/or therapeutic agents contained in the fillers are released by dehydration or degradation mechanisms.

In accordance with certain exemplary embodiments of the present invention, adjuvants or fillers may be added to the composition or combination of materials. Adjuvants or filler materials can be chosen in order to allow bonding between the signal-generating agents or the therapeutic agents and the implant material, and/or to allow bonding between two or more agents. Adjuvants and fillers may further assist in the material bonding of the composition or combination of materials by physical or chemical mechanisms, or alternatively to modulate the elasto-mechanical, chemical, or biological properties thereof. The adjuvants and/or fillers may be chosen in the form of micelles, microspheres, macrospheres, liposomes, nano-, micro- or macro-capsules, microbubbles, etc. They may also be present in the form of functional units, for example by attachment of appropriate functional groups and compounds thereto. The adjuvants and fillers may further be selected to assist in attaching a composition used in forming a component of an implantable medical device to another component or part of the implantable device, including for example to improve the adhesion of a coating to an underlying substrate material.

Adjuvants may comprise polymer, non-polymer, organic, inorganic, or composite materials. Alteration of elasto-mechanical properties can be achieved by adding fibers made of carbon, polymer, glass, or other materials, of any size and in woven or non-woven form.

Adjuvants may also be used to modulate material behavior, for example to retard the release of signal generating and/or therapeutic agents. Adjuvants may be selected based on the purpose and location of insertion of the implantable medical device, and they may comprise a degradable or non-degradable material and/or a hydrophobic or hydrophilic material or any desired mixture thereof. Adjuvants may also have crystalline, semi-crystalline or amorphous forms.

The degradation rate and/or release of agents from partially degradable or degradable or non-degradable devices in the physiological medium may be adjusted by, for example, the mixing of hydrophobic and hydrophilic adjuvants. Also, the predominant presence of crystalline, semi-crystalline or amorphous phases, or of mixtures of hydrophobic and hydrophilic substances, may be adjusted by selection of substances based at least in part on their melting points. For example, polymers may be selected which have melting points close to, above, or below the body temperature of the target organism. The solubility of the adjuvants, which may exist as matrix material, micelles, microspheres, liposomes or capsules or similar structures, may thus be varied within the target organism, and thereby affect or control the elution, erosion or degradation of the agents or of the medical devices themselves. In a further exemplary embodiment, the solids content of the adjuvants may be adjusted, which may thereby influence or control the desired leaching, release or degradation rates therewith. For example, coating thicknesses or matrix volumes may be adjusted to produce desired degradation rates.

In other exemplary embodiments of the present invention, an implantable medical device, such as a metallic stent or a pacemaker electrode or an artificial heart valve, may be coated with a porous coating, for example with a pyrolytic carbon coating such as that described in German publication DE 202004009060U. The coating may be subsequently provided or loaded with at least one signal-generating agent as described above, and simultaneously or subsequently with at least one therapeutic agent as described above, wherein these agents are selected in accordance with the intended use of the device and the loading order of the different agents may be selected as appropriate to achieve the desired results. The loading may be achieved by spraying, impregnating with solutions, or in any other suitable way. If necessary, further adjuvants or overcoatings may be applied in order to control the release rates of the agents. The average release rates of the signal-generating agent and the therapeutic agent from implantable devices produced in this manner may be determined by common in-vitro tests performed in a balanced salt solution or in any other suitable media. From concentration measurements, which may be optionally combined with non-invasive physical detection methods for the signal-generating agents, a correlation coefficient for the amount of therapeutic agent released per amount of signal intensity obtained from the signal-generating agent can be determined for a given combination of agents, which allows for an indirect determination of the amount of therapeutic agent released in relation to the signal intensity obtained by detecting the signal-generating agent. By using this method, monitoring of the amount and the regional distribution of released therapeutic agent is made possible through the use of simple, non-invasive physical detection methods. The invention is now further explained in the following examples, in order to represent the principle of the composition or combination described above in some exemplary embodiments of the present invention. These examples are merely illustrative and do not indicate any necessary limitations to the present invention.

### EXAMPLES

### Example 1

A commercially available, X-ray dense, non-fluorescing coronary stent from Fortimedix Company (KAON Stent), Netherlands, 18.5 mm long, and made of stainless steel 316L was coated with a coating of carbon-Si composite material in accordance with German Patent No. DE 202004009060U. A phenoxy resin obtained from UCB Company, Beckopox EP 401, was used as a precursor polymer, and a dispersion of commercially available Aerosil R972 (obtained from Degussa) in methylethylketone was prepared. The solids content of the polymer amounted to 0.75 wt %, the solids content of Aerosil in the dispersion was 0.25 wt %, and the solids content of solvent was 99 wt %. The precursor solution was sprayed onto the substrate as a polymer film and tempered by application of hot air at 350° C. in ambient air. The crude weight of the polymer film was subsequently determined, and the coating was found to have a surface area weight of about 2.53 g/m². The sample was then examined in a Nikon fluorescence microscope for its inherent fluorescence. The crude coating did not exhibit any fluorescence. Subsequently the sample was treated thermally in a commercial tube reactor, in accordance with the disclosure of German Patent No. DE 202004009060U. The thermal treatment was carried out under a nitrogen atmosphere with a heat-up and cool-down ramp of 1.3 K/min, a holding temperature of 300° C., and a holding period time of 30 minutes. Subsequently the sample was treated in an ultrasonic bath in 10 ml of a 50% ethanol solution at 30° C. for 20 minutes, washed, and dried in a commercial convection oven at 90° C. The gravimetric analysis indicated a shrinkage after the thermal treatment of about 29% and a surface area weight of the composite layer of glassy, amorphous carbon/Si of 1.81 g/m². A scanning electron microscope investigation revealed a porous layer having an average pore diameter of about 100 nm. A subsequent investigation in a fluorescence microscope showed an intense fluorescence of the coated coronary stent in the green and blue regions, as well as a weak fluorescence in the red region.

### Example 2

As in Example 1, a commercially available, X-ray dense, non-fluorescing coronary stent from Fortimedix Company (KAON Stent), Netherlands, 18.5 mm long and made of 316L stainless steel was coated with a coating of carbon-Si composite material in accordance with the disclosure of German Patent No. DE 202004009060U. The composition of the precursor in this example was modified to modify the fluorescence emission spectrum in the red region. A phenoxy resin from UCB Company, Beckopox EP 401, was used as the precursor polymer, and it was combined with a dispersion of commercially available Aerosil R972 (from Degussa) in methylethylketone. Additionally, isophorone diisocyanate (from Sigma Aldrich Company) was introduced as a cross-linking agent. The solids content of the polymer amounted to 0.55 wt %, the solids content of Aerosil was 0.25 wt %, the solids content of the cross-linking agent was 0.2 wt %, and the solid portion of solvent was 99 wt %. The precursor solution was sprayed onto the substrate as a polymer film, tempered by application of hot air at 350° C. in ambient air, and subsequently the crude weight of the polymer film determined. The coating was found to have a surface area weight of about 2.20 g/m². The sample was subsequently examined in a Nikon fluorescence microscope for its inherent fluorescence. The crude coating did not exhibit any fluorescence. Subsequently the sample was treated thermally in a commercial tube reactor, in accordance with the disclosure of German Patent No. DE 202004009060U. The thermal treatment was carried out under a nitrogen atmosphere with a heat-up and cool-down ramp of 1.3 K/min, a holding temperature of 300° C., and a holding period of 30 minutes.

The sample was then treated in an ultrasonic bath in 10 ml of a 50% ethanol solution at 30° C. for 20 minutes, washed, and dried in a commercial convection oven at 90° C. The gravimetric analysis indicated a shrinkage after the thermal treatment of about 23% and a surface area weight of the composite layer of glass-like amorphous carbon/Si of 1.69 g/m². The scanning electron microscope investigation revealed a porous layer having an average pore diameter of about 100 nm. A subsequent investigation in a fluorescence microscope showed an intensive fluorescence of the coated coronary stent in the green and blue regions, as well as a strong fluorescence in the red region.

### Example 3

The coronary stents produced in Example 1 and Example 2 above were subsequently charged with an active agent. Paclitaxel, obtained from Sigma Aldrich, was used as model substance. A Paclitaxel solution having a concentration of 43 g/l was prepared in ethanol. The stents were subjected to a gravimetric analysis before and after being charged by dipping in 5 ml of the ethanolic paclitaxel solution. The charge was carried out by dipping the stent in the active agent solution for 10 minutes. The overall charge was determined from the increase in mass after the dipping step. The sample from Example 1 had a loading of 0.766 g/m², and the sample from Example 2 had a loading of 0.727 g/m². After drying each stent in air for 60 minutes, another fluorescence microscopy investigation was carried out, which showed the same fluorescence characteristics as was observed for the unloaded porous coatings (strong blue and green fluorescence and weak red fluorescence sample for the stent of Example 1, and strong red fluorescence for the stent of Example 2).

### Example 4

Three commercially available, X-ray dense, non-fluorescing coronary stents from Fortimedix Company (KAON Stent), Netherlands, 18.5 mm long, made of 316L stainless steel were coated with a coating of carbon-carbon composite material in accordance with the disclosuyr eof German Patent No. DE 202004009060U. A phenoxy resin, Beckopox EP 401 (from UCB Company), was used as a precursor polymer. A dispersion was prepared of this polymer, commercially available carbon black, Printex alpha (from Degussa), and a fullerene mixture of C60 and C70 (from FCC Company, sold as Nanom-Mix), in methylethylketone. The solids content of the polymer amounted to 0.5 wt %, the solids content of carbon black was 0.3 wt %, the solids content of the fullerene mix was 0.2 wt %, and the solvent accounted for 99 wt % of the dispersion. The precursor solution was sprayed onto the substrate as a polymer film and tempered by application of hot air at 350° C. in ambient air. The crude weight of the polymer film was then determined, and the coating was found to have a surface area weight of about 2.5 g/m². The sample was subsequently examined in a Nikon fluorescence microscope for its inherent fluorescence. The crude coating did not exhibit any fluorescence. Subsequently the sample was treated thermally in a commercial tube reactor, in accordance with the disclosure of German Patent No. DE 202004009060U. The thermal treatment was carried out under nitrogen atmosphere with a heat-up and cool-down ramp of 1.3 K/min, a holding temperature of 300° C., and a holding period of 30 minutes. The sample was then treated in an ultrasonic bath in 10 ml of a 50% ethanol solution at 30° C. for 20 minutes, washed, and dried in a commercial convection oven at 90° C. The gravimetric analysis indicated a shrinkage after the thermal treatment of about 30%, and a surface area weight of the composite coating of a glass-like amorphous carbon/pyrolytic carbon of 1.75 g/m². The scanning electron microscope revealed an average pre size of about 1 μm. A fluorescence microscopic investigation indicated no fluorescence of the coating.

To load an active agent, a 1 mM Calcein-AM-solution in DMSO (from Mobitec Company) was first diluted to a 1:1000 ratio in acetone. Subsequently, 0.5 mg of the calcein solution was mixed together with 20 mg of poly(DL-lactide coglycolide) and 2 mg Paclitaxel in 3 ml of acetone. The resulting solution was added with a constant flow rate of 10 ml/min to a solution of 0.1% Poloxamer 188 (pluronic F68) in 0.05 M PBS buffer while stirring at 400 rpm. This colloidal suspension was stirred further for 3 hours under light vacuum for evaporation of the solvents, and subsequently dried completely for 14 hours under full vacuum. The nanoparticles thus obtained with encapsulated Paclitaxel and in-vivo fluorescence marker were subsequently re-suspended in ethanol and the concentration of the particle-containing solution was obtained by determining the solids content.

The three coated coronary stents were subsequently loaded with the nanoparticles by dipping, and the charged weight was determined gravimetrically. The average loading of the convection oven-dried coronary stents amounted to 0.5±0.05 g/m². Subsequently, the expanded stents were introduced into 6 well-plates and incubated with about 10⁵ cells/ml of three times passaged COS-7 cell cultures (37.5° C., 5% CO₂) in DMEM medium in a culture volume of 5 ml. For each stent, measurements were taken of the culture volumes and the released amounts determined by means of HPLC immediately after the expansion, after 1, 3, 6, 12, 24, and 36 hours, and after 2, 3, 4, 5, 7, 9, 12, 15, 21 and 30 days. The medium was replaced after each measurement. Further, the samples were investigated in the fluorescence microscope and the adherent cells investigated for fluorescence in the green region. By means of Lucia software from Nikon Company, an area of 0.5 μm² was analyzed in each measurment by means of the densitometric measurement of the average color intensity of the fluorescence intensity. The densitometric maximum was observed after 30 days, and the correlation between intensity of the fluorescence values and the release of calcein-AM in was determined as a percentile versus time.

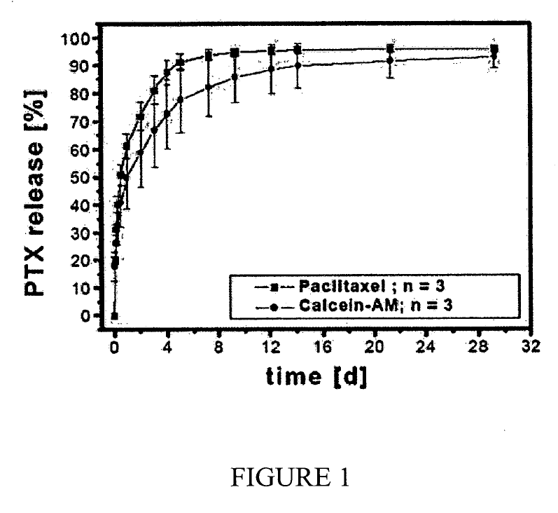

The graph of FIG. 1 shows the measured correlation between the release of adsorbed Paclitaxel from the encapsulated nanoparticles of the coronary stent and the in-vivo activity of the fluorescent coloring of Calcein-AM. After a period of 35 days the samples were transferred into new culture vessels and incubated with fresh cell suspensions. Paclitaxel could not be identified in the new medium, nor was there any fluorescence coloring of the cell culture.

Having thus described in detail several exemplary embodiments of the present invention, it is to be understood that the exemplary embodiments of the present invention recited in the claims below are not to be limited to particular details set forth in the above description, as many apparent variations thereof are possible without departing from the spirit or scope of the present invention.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any document in this application is not an admission that such document is available as prior art to the present invention. It is noted that in this disclosure and particularly in the claims, terms such as “comprises,” “comprised,” “comprising” and the like can have the meaning attributed to them in U.S. Patent law; e.g., they can mean “includes,” “included,” “including” and the like; and that terms such as “consisting essentially of” and “consists essentially of” can have the meaning ascribed to them in U.S. Patent law, e.g., they allow for elements not explicitly recited, but exclude elements that are found in the prior art or that affect a basic or novel characteristic of the invention. The embodiments of the present invention are disclosed herein or are obvious from and encompassed by the detailed description. The detailed description, given by way of example, but not intended to limit the invention solely to the specific embodiments described, may best be understood in conjunction with the accompanying Figure.

What is claimed is:

1. A composition for use in an implantable medical device, comprising:
at least one first agent capable of causing a detection of signals by at least one of a physical measurement, a biological measurement, a chemical measurement, or a verification method;
at least one material for manufacture of at least one portion of the implantable medical device; and
at least one second agent capable of causing a therapeutic action in at least one of an animal organism or a human organism.

2. The composition of claim 1, wherein the second agent is capable of being released one of directly or indirectly into at least one of an animal organism or a human organism from the at least one portion of the implantable medical device.

3. The composition of claim 1, wherein the first agent has at least one additional property in addition to the causing the detection of the signals.

4. The composition of claim 1, wherein the first agent is capable of causing a detection of signals in the absence of at least one of a physical stimulus, a chemical stimulus, a biological stimulus, and a physiologically conditioned in-vivo change.

5. The composition of claim 1, wherein the first agent is capable of causing the detection of the signals in response to at least one of a physical stimulus, a chemical stimulus, or a biological stimulus.

6. The composition of claim 1, wherein the first agent is capable of causing a detection of signals in response to at least one of a physical change in-vivo, a chemical change in-vivo, a biological change in-vivo, or a physiologically-conditioned change in-vivo.

7. The composition of claim 1, wherein the at least one material comprises biologically degradable materials.

8. The composition of claim 1, wherein the at least one material comprises biologically non-degradable materials.

9. The composition of claim 1, wherein the at least one material comprises a combination of biologically non-degradable materials and biologically degradable materials.

10. The composition of claim 3, wherein the at least one additional property is capable of causing one of a direct therapeutic action or an indirect therapeutic action in at least one of an animal organism or a human organism.

11. The composition of claim 3, wherein the at least one additional property comprises a capability to provide at least one targeting group.

12. The composition of claim 3, wherein the first agent comprises a first unit and a second unit which are covalently bonded to each other, wherein the second unit has at least one property that is different from a property which causes the detection of the signals by at least one of a physical measurement, a biological measurement, a chemical measurement, or a verification method.

13. The composition of claim 12, wherein the at least one property comprises at least one of causing a therapeutic action in at least one of an animal organism or a human organism, or providing a targeting group.

14. The composition of claim 13, wherein the first agent further comprises a third unit that includes at least one of an agent capable of causing a therapeutic action in at least one of an animal organism or a human organism, or a targeting group.

15. The composition of claim 3, wherein the first agent comprises a first unit and a second unit which are non-covalently bonded to each other, and wherein the second unit has at least one property that is different than a property which is capable of causing the detection of the signals by at least one of a physical measurement, a biological measurement, a chemical measurement, or a verification method.

16. The composition of claim 15, wherein the at least one property provides at least one of a therapeutically active agent or a targeting group.

17. The composition of claim 16, wherein the first agent further comprises a third unit that includes at least one of a therapeutically active agent or a targeting group.

18. The composition of claim 1, further comprising at least one region exhibiting a concentration gradient in a local distribution of the first agent.

19. The composition of claim 1, wherein the composition is a coating capable of being applied on at least one portion of the implantable medical device, wherein the coating further comprises a first layer and a second layer, and wherein a concentration of the first agent in the first layer differs from a concentration of the first agent in the second layer.

20. The composition of claim 1, further comprising at least one adjuvant.

21. The composition of claim 20, wherein the adjuvant is a polymer.

22. The composition of claim 20, wherein the adjuvant is a non-polymeric material.

23. The composition of claim 20, wherein the adjuvant is an inorganic material.

24. The composition of claim 20, wherein the adjuvant is an organic material.

25. The composition of claim 20, wherein the adjuvant comprises an inorganic-organic composite material.

26. The composition of claim 20, wherein the adjuvant is biodegradable.

27. The composition of claim 20, wherein the adjuvant is non-degradable.

28. The composition of claim 20, wherein the adjuvant is partially biodegradable.

29. The composition of claim 20, wherein the adjuvant is capable of controlling a release of at least one of the first agent or the second agent when the composition is at least one of exposed to physiologic fluids or implanted into at least one of a human organism or an animal organism.

30. The composition of claim 1, further comprising a third agent capable of causing a detection of further signals by at least one of a further measurement method or a further verification method, wherein the first agent is approximately precluded from causing a detection of signals by the at least one of a further measurement method or a further verification method.

31. The composition of claim 30, wherein the first agent is capable of causing the detection of the signals by at least one of a conventional X-ray method, an X-ray-based split-image method including computer tomography, a neutron transmission tomography procedure, a radio frequency magnetization procedure including magnetic resonance tomography, a method based on radio nuclides including scintigraphy, a single photon emission computed tomography (SPECT) procedure, a positron emission computed tomography (PET) procedure, an ultrasonic-based method, a fluoroscopic method, a luminescence or fluorescence-based method including intravasal fluorescence spectroscopy, a Raman spectroscopy procedure, a fluorescence emission spectroscopy procedure, an electrical impedance spectroscopy procedure, a colorimetry procedure, an optical coherence tomography procedure, an electron spin resonance (ESR) procedure, a radiofrequency (RF) method, or a microwave laser method.

32. The composition of claim 31, wherein the third agent agent is capable of causing a detection of signals by at least one of a conventional X-ray method, an X-ray-based split-image method including computer tomography, a neutron transmission tomography procedure, a radio frequency magnetization procedure including magnetic resonance tomography, a method based on radio nuclides including scintigraphy, a single photon emission computed tomography (SPECT) procedure, a positron emission computed tomography (PET) procedure, an ultrasonic-based method, a fluoroscopic method, a luminescence or fluorescence-based method including intravasal fluorescence spectroscopy, a Raman spectroscopy procedure, a fluorescence emission spectroscopy procedure, an electrical impedance spectroscopy procedure, a colorimetry procedure, an optical coherence tomography procedure, an electron spin resonance (ESR) procedure, a radiofrequency (RF) method, or a microwave laser method.

33. The composition of claim 30, wherein at least one of the first agent or the third agent is selected from the group consisting of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts including salts and chelates from the lanthanide group with atomic numbers 57-83 or from the transition metals with atomic numbers 21-29, 42 or 44, metal polymers, metallocenes, or other organometallic compounds including metal complexes with phthalocyanines.

34. The composition of claim 30, wherein at least one of the first agent or the third agent is selected from the group consisting of magnetic materials including those with paramagnetic, diamagnetic, super paramagnetic, ferrimagnetic or ferromagnetic properties; semiconducting materials including those from the Groups II-VI, Groups III-V, or Group IV, having absorption properties for radiation in wavelength ranges approximately from gamma rays up to microwave radiation and/or emitting radiation properties.

35. The composition of claim 30, wherein at least one of the first agent or the third agent is selected from the group consisting of ionic and non-ionic halogenated agents including 3-acetylamino-2,4-6-triiodobenzoic acid, 3,5-diacetamido-2,4,6-triiodobenzoic acid, 2,4,6-triiodo-3,5-dipropionamidobenzoic acid, 3-acetyl amino-5-((acetyl amino)methyl)-2,4,6-triiodobenzoic acid, 3-acetyl amino-5-(acetylmethylamino)-2,4,6-triiodobenzoic acid, 5-acetamido-2,4,6-triiodo-N-((methylcarbamoyl)methyl)isophthalamic acid, 5-(2-methoxyacetamido)-2,4,6-triiodo-N-[2-hydroxy-1-(methylcarbamoyl)-ethyl]-isophthalamic acid, 5-acetamido-2,4,6-triiodo-N-methylisophthalamicacid, 5-acetamido-2,4,6-triiodo-N-(2-hydroxyethyl)isophthalamic acid, 2-[[2,4,6-triiodo-3[(1-oxobutyl)amino]phenyl]methyl]butanoic acid, beta-(3-amino-2,4,6-triiodophenyl)-alpha-ethylpropionic acid, or iopamidol, iotrolan, iodecimol, iodixanol, ioglucol, loglucomide, iogulamide, iomeprol, or iopentol.

36. The composition of claim 30, wherein at least one of the first agent or the third agent is selected from the group consisting of carbon species including carbides, fullerenes, fullerene-metal complexes, or endohedral fullerenes which contain rare earths including cerium, neodymium, samarium, europium, gadolinium, terbium, dysprosium or holmium, or halogenated fullerenes.

37. The composition of claim 30, wherein at least one of the first agent or the third agent is selected from the group consisting of anionic and/or cationic lipids, including halogenated anionic or cationic lipids.

38. The composition of claim 30, wherein at least one of the first agent or the third agent is selected from the group consisting of gases or in-vivo gas-forming substances including air, nitrogen, hydrogen, alkanes, or halogenated hydrocarbon gases including methyl chloride, perfluoroacetone, and perfluorobutane.

39. The composition of claim 38, wherein at least one of the gases or the in-vivo gas-forming substances are contained in at least one of microbubbles or microspheres.

40. The composition of claim 30, wherein at least one of the first agent or the third agent is selected from the group consisting of recombinant and non-recombinant nucleic acids, proteins, peptides, or polypeptides, including those which directly or indirectly induce the in-vivo formation or enrichment of signal-generating agents and those which contain coding sequences for the expression of signal-generating agents including metallo-protein complexes, dicarboxylate proteins, lactoferrin or ferritin, or those that regulate enrichment and/or homeostasis of physiologically available signal-generating agents such as the iron regulatory protein (IRP), transferrin receptor, or erythroid 5-aminolevulinate synthase.

41. The composition of claim 30, wherein at least one of the first agent or the third agent is provided in a form of at least one of polymeric nanoparticles, non-polymeric nanoparticles, or microparticles, wherein an average size of the polymeric nanoparticles, the non-polymeric nanoparticles, or the microparticles is between about 2 nm and 20 μm.

42. The composition of claim 41, wherein the average size of the polymeric nanoparticles, the non-polymeric nanoparticles, or the microparticles is between about 2 nm to 5 μm.

43. The composition of claim 30, wherein at least one of the first agent or the third agent is provided in a form of at least one of microspheres, macrospheres, micelles or liposomes, or encapsulated in polymeric shells.

44. The composition of claim 30, wherein at least one of the first agent or the third agent is provided in a form of biological vectors, including transfection vectors such as virus particles or viruses, including adeno viruses, adeno virus associated viruses, herpes simplex viruses, retroviruses, alpha viruses, pox viruses, arena-viruses, vaccinia viruses, influenza viruses or polio viruses.

45. The composition of claim 30, wherein at least one of the first agent or the third agent comprises at least one of cells, cell cultures, organized cell cultures, tissues, organs of any desired species, or non-human organisms, and wherein the at least one of the first agent or the third agent further comprises recombinant nucleic acids with coding sequences capable of producing at least one agent capable of causing the detection of the signals by at least one of a physical measurement, a biological measurement, a chemical measurement, or a verification method.

46. The composition of claim 30, wherein at least one of the first agent or the third agent is provided in a form of at least one of a solution, a suspension, an emulsion, a dispersion, or a solid material.

47. The composition of claim 30, wherein the first agent is bonded covalently to the third agent.

48. The composition of claim 30, wherein the first agent is bonded non-covalently to the third agent.

49. An implantable medical device comprising:
at least one first agent capable of causing a detection of signals by at least one of a physical measurement, a biological measurement, a chemical measurement, or a verification method;
at least one material for manufacture of at least one portion of the implantable medical device; and
at least one second agent capable of causing a therapeutic action in at least one of an animal organism or a human organism.

50. The implantable medical device of claim 49, wherein the at least one material comprises at least one polymer selected from the group consisting of polyurethane, collagens, albumin, gelatin, hyaluronic acid, starch, cellulose (methylcellulose, hydroxypropylcellulose, hydroxypropylmethylcellulose, carboxymethylcellulosephthalat, casein, dextrane, polysaccharides, fibrinogen, poly(D,L-lactide), poly(D,L-lactide-co-glycolide), poly(glycolides), poly(hydroxybutylate), poly(alkyl carbonates), poly(orthoesters), polyesters, poly(hydroxyvaleric acid), polydioxanone, poly(ethylene terephthalate), poly(malic acid), poly(tartronic acid), polyanhydrides, polyphosphohazenes, poly(amino acids).

51. The implantable medical device of claim 49, wherein the at least one material comprises at least one non-polymeric materials selected from the group consisting of ceramics, glasses, metals, alloys, bone, stone, minerals, or any mixture thereof.

52. The implantable medical device of claim 49, wherein the at least one material comprises a mixture of non-polymeric and polymeric materials.

53. The implantable medical device of claim 49, wherein the at least one material comprises magnesium or zinc.

54. The implantable medical device of claim 53, wherein the medical device is a stent.

55. The implantable medical device of claim 54, wherein the stent is at least partially coated with a coating comprising particles containing at least one of magnesium or zinc.

56. The implantable medical device of claim 49, wherein the first agent is present in a porous reticulated network which is capable of being loaded with the second agent.

57. A method for determining of the extent of release of a therapeutically active agent from an implantable medical device, comprising:
providing the implantable medical device comprising at least one first agent capable of causing a detection of signals by at least one of a physical measurement, a biological measurement, a chemical measurement, or a verification method, and at least one second agent capable of causing a therapeutic action in at least one of an animal organism or a human organism, wherein the medical device is capable of at least partially releasing the second agent together with the first agent after insertion of the device into at least one of a human organism or an animal organism;
determining a correlation between an amount of the second agent released and an amount of the first agent released;
detecting an extent of release of the first agent through the application of at least one of a non-invasive measurement or a verification method; and
determining an extent of release of the second agent by applying the correlation.

58. The method of claim 57, wherein the implantable medical device is at least partially degradable, and wherein the first and second agents are released during the at least partial degradation of the medical device.

59. The method of claim 57, wherein the implantable medical device is non-degradable.

60. The method of claim 57, wherein the first agent is covalently bonded to the second agent.

61. The method of claim 57, wherein the first agent is non-covalently bonded to the second agent.
